(19) United States
(12) Patent Application Publication
(10) Pub. No.: US 2026/0364187 A1
(43) Pub. Date: Nov. 19, 2026

(54) METHODS OF MAKING SITE SPECIFIC ACTIVATED ANTIBODIES AND ANTIBODY CONJUGATES

(71) Applicant: Life Technologies Corporation, Carlsbad, CA (US)

(72) Inventors: Byron Hetrick, Eugene, OR (US); Robert Aggeler, Eugene, OR (US); Yi-Zhen Hu, Eugene, OR (US); Brian Agnew, Eugene, OR (US); Thaís de Faria, Eugene, OR (US)

(21) Appl. No.: 19/660,154

(22) Filed: Apr. 27, 2026

Related U.S. Application Data
(60) Provisional application No. 63/796,594, filed on Apr. 29, 2025.

(51) Int. Cl. A61K 47/68; C07K 16/00; C12N 9/10; C12N 9/38

(52) U.S. Cl. CPC A61K 47/68; C07K 16/00; C12N 9/1051; C12N 9/2471; C07K 2317/41; C12Y 302/01023

(57) ABSTRACT

Improved methods are provided for making activated antibodies and antibody conjugates that combine the necessary enzymatic reactions into a single step resulting in a faster and more efficient process. The present disclosure provides methods for making activated antibodies and antibody conjugates with a degree of substitution (DOS) of about 4 as well as methods for making activated antibodies and antibody conjugates with a DOS of about 2. Also provided are activated antibodies and antibody conjugates made by the single step workflows of the present disclosure. Kits for making activated antibodies and antibody conjugates according to the single step workflows of the present disclosure are also provided.

### CROSS-REFERENCE

This application claims the benefit under 35 U.S.C. § 119(e) of U.S. Provisional Application No. 63/796,594, filed Apr. 29, 2025. The entire contents of the aforementioned application(s) are incorporated by reference herein

### FIELD

The present disclosure is generally directed to methods of making site-specific activated antibodies and antibody conjugates, including methods for attaching biologically or pharmaceutically active molecules to antibodies for developing reagents such as antibody-drug conjugates, antibody labeling reagents, and the like.

### BACKGROUND

Antibody conjugates have been used in the sensitive and multiplexed detection of proteins in single cells or cell populations and also as a platform for targeted drug delivery for treating a variety of diseases. For example, antibodies can be conjugated to a variety of compounds including small-molecule drugs, fluorophores, catalysts, synthetic polymers, oligonucleotides, peptides, enzymes and other proteins, for use as biological tools or as diagnostic or therapeutic agents. All IgGs have an N-linked glycan on asparagine 297 (N297 or Asn297) in the Fc domain, and this residue has been used to synthesize site-specific antibody conjugates. Current platforms utilize glycan modifying enzymes to chemoenzymatically synthesize antibody conjugates via Fc glycan remodeling and require prolonged incubation times resulting in workflows that span multiple hours to days. Installing a macromolecule via these platforms usually follows a two-step activation procedure in which bioorthogonal reaction handles, such as azides, are introduced onto the Fc region via unnatural glycosylation, followed by a third step wherein a biologically or pharmaceutically active molecule is further installed by bioorthogonal reactions.

Currently, existing methods for antibody conjugation involve multiple steps and require prolonged workflow times. A need, therefore, exists to provide a more streamlined workflow that significantly reduces the time required for antibody modification.

### SUMMARY

One embodiment of the present disclosure provides a method comprising contacting an antibody with a modified sugar substrate comprising a first chemical handle, a mutant β-1,4-galactosyltransferase, and a β-galactosidase in a single step to form an activated antibody. The method can further comprise contacting the activated antibody with a biologically or pharmaceutically active molecule that comprises a second chemical handle that reacts with the first chemical handle to form an antibody conjugate. In certain embodiments, the degree of labeling is about 4.

Another embodiment of the present disclosure provides a method comprising contacting an antibody with a modified sugar substrate comprising a first chemical handle, a mutant β-1,4-galactosyltransferase, and an endoglycosidase in a single step to form an activated antibody. The method can further comprise contacting the activated antibody with a biologically or pharmaceutically active molecule that comprises a second chemical handle that reacts with the first chemical handle to form an antibody conjugate. In certain embodiments, the degree of labeling is about 2.

In certain embodiments, the first chemical handle comprises an azide group, an alkyne group, a cycloalkene group, a ketone group, an oxime group, a nitrile oxide group, or a vinyl group. In certain embodiments, the modified sugar substrate is chosen from UDP-GalNAz, UDP-GalKyne, UDP-Gal-Cyclopropene, UDP-GalKetone, UDP-Gal-Oxime, UDP-Gal-Nitrile oxide, UDP-GalVinyl, UDP-GalVinylSulfo, and UDP-GalVinylPhospho. In certain preferred embodiments, the modified sugar substrate is UDP-GalNAz. In certain embodiments, the second chemical handle comprises an alkyne-reactive group, an azide-reactive group, a Diels-Alder diene, a Diels-Alder dienophile, a straight chain or branched C₁-C₁₂ carbon chain bearing a carbonyl group, a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR³NH₂ (carbazide), —NR¹NR²(C═S)NR³NH₂ (thiocarbazide), —ONH₂ (aminooxy), wherein each R¹, R², and R³ is independently H or alkyl having 1-6 carbons, or a thiol. In certain preferred embodiments, the second chemical handle comprises a strained ring alkyne. In certain embodiments, the mutant β-1,4-galactosyltransferase is a Y289L, Y289N, Y289I, Y289F, Y289M, Y289V, Y289G, or Y289A mutant β-1,4-galactosyltransferase. In certain preferred embodiments, the mutant β-1,4-galactosyltransferase is a Y289L mutant β-1,4-galactosyltransferase. In certain embodiments, the biologically or pharmaceutically active molecule is chosen from a fluorescent dye, an enzyme, a radiolabel, a metal chelator, a detectable substrate, a therapeutic agent, a nucleic acid, a polynucleotide, an oligonucleotide, a protein, a peptide, a sugar, an oligosaccharide, a lipid nanoparticle, and a bead. In certain embodiments, the endoglycosidase is chosen from Endo S, Endo A, Endo F, Endo M, Endo D, and Endo H, or a combination thereof. In certain preferred embodiments, the endoglycosidase is Endo S or Endo S2.

Yet another embodiment of the present disclosure provides an activated antibody made by a method of the present disclosure. In certain embodiments, the present disclosure provides an antibody conjugate made by a method of the present disclosure.

Yet another embodiment of the present disclosure provides a kit for making an activated antibody or antibody conjugate according to methods of the present disclosure. In certain embodiments, the present disclosure provides a kit for making an activated antibody or antibody conjugate according to methods of the present disclosure.

The foregoing has outlined rather broadly the features and technical advantages of the present disclosure in order that the detailed description of the disclosure that follows may be better understood. Additional features and advantages of the present disclosure will be described hereinafter which form the subject of the claims. It should be appreciated by those skilled in the art that the conception and specific embodiments disclosed may be readily utilized as a basis for modifying or designing other structures for carrying out the same purposes of the present disclosure. It should also be realized by those skilled in the art that such equivalent constructions do not depart from the spirit and scope of the present disclosure as set forth in the appended claims. The novel features which are believed to be characteristic of the present disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1A illustrates an existing three-step antibody conjugation method which uses a two-step antibody activation workflow resulting in a degree of labeling (DOL) of about 4. This method is referred to herein as “Classic SiteClick™”.

FIG. 1B illustrates an exemplary two-step antibody conjugation method which uses a one-step antibody activation workflow resulting in a degree of labeling (DOL) or degree of substitution (DOS) of about 4 in accordance with one embodiment of the present disclosure. This method is referred to herein as “One-Step DOS4”.

FIG. 1C illustrates an exemplary two-step antibody conjugation method which uses a one-step antibody activation workflow resulting in a degree of labeling (DOL) or degree of substitution (DOS) of about 2 in accordance with one embodiment of the present disclosure. This method is referred to herein as “One-Step DOS2”.

FIG. 2 shows a flow-chart of an exemplary one-step antibody activation workflow and the follow-on antibody conjugation workflow, such as that described in Example 2.

FIG. 3 shows a flow-chart of an exemplary one-step antibody activation workflow and the follow-on antibody conjugation workflow, such as that described in Example 5.

FIGS. 4A-4C show β-galactosidase activity at pH 6 and 7. FIG. 4A shows the pH dependence of the rate of β-galactosidase cleavage reaction on a small molecule substrate, DDAO-galactoside, at pH 6 and 7. FIG. 4B shows the stability of the β-galactosidase activity to overnight (16 h) incubation at 37 °C at pH 7. FIG. 4C shows the pH dependence of the rate of β-galactosidase cleavage on an antibody substrate measured by mass spectrometry. The graph displays the percentage of total NIST mAb that has a GOF/GOF glycan quantified as a function of time incubated with β-galactosidase.

FIGS. 5A-5D demonstrate that the One-Step DOS4 workflow can modify fucosylated antibodies. FIG. 5A shows the mass spectra of NIST mAb treated with β-galactosidase (top) or unmodified (bottom).

FIG. 5B shows the distribution of glycan species in the NIST mAb with and without β-galactosidase treatment. FIG. 5C shows a comparison of mass spectra of NIST mAb treated with the One-Step DOS4 workflow (top) or β-galactosidase treatment alone (bottom). FIG. 5D shows the distribution of activated antibody species with different degrees of GalNAz substitution resulting from the One-Step DOS4 workflow.

FIG. 6 shows that the One-step DOS4 workflow results in equivalent labeling as the Classic SiteClick workflow. FIG. 6 shows a graph comparing the degree of labeling (DOL) of NIST mAb by sDIBO-AF488 after azide modification of the antibody glycan according to the indicated workflow (No Beta-Gal, Classic SiteClick, or One-step DOS4).

FIGS. 7A-7B show that the One-Step DOS4 workflow allows labeling across a variety of antibody isotypes. FIG. 7A shows a graph depicting the degree of labeling (DOL) by sDIBO-AF488 after preparation of the indicated antibody isotypes using the One-Step DOS4 workflow. FIG. 7B shows images of SDS-PAGE of antibody isotypes showing total protein (left) and AF488 labeled products (right). The identities of antibodies used corresponding to each isotype are indicated in Table 2.

FIGS. 8A-8C show flow cytometry data indicating the One-Step DOS4 modified antibodies are functional in flow cytometry. Human PBMCs were stained Rituximab (CD20) conjugated to sDIBO-AF488 using the One-Step DOS4 workflow. Rituximab-AF488 staining was mutually exclusive to CD3 eF450 staining and staining intensity varied with the amount of antibody used in staining: 1.0 g (FIG. 8A), 0.5 g (FIG. 8B), or 0.25 g (FIG. 8C).

FIGS. 9A-9B show antibodies modified by the One-Step DOS2 workflow are stable to further Endo S cleavage. FIG. 9A (right) shows the mass spectra of CD28 clone 28.2 monoclonal antibody (mAb) treated with the One-Step DOS2 workflow before (bottom) and after (top) subsequent treatment with additional Endo S. The mass spectrometry profile shows no loss of the GalNAz modification. FIG. 9A (left) shows a schematic of the azide-activated antibody. FIG. 9B (right) shows the UV-Vis absorbance spectrum of CD28 clone 28.2 mAb treated with the One-Step DOS2 workflow followed by labeling with sDIBO-AF488 and treated with additional Endo S. The UV-Vis spectrum indicates the dye was unable to be removed by further Endo S treatment. FIG. 9B (left) shows a schematic of the labeled activated antibody.

FIGS. 10A-10B show that the One-Step DOS2 workflow can modify and label fucosylated antibodies. FIG. 10A shows the mass spectra of NIST mAb unmodified (bottom) and after Endo S treatment (top). The NIST-mAb digested with Endo S shows peaks with variable fucosylation states. FIG. 10B shows the mass spectra of CD28 clone 28.2 mAb after the treatment with the One-Step DOS 2 workflow (bottom) and after the treatment with Endo S alone (top). The bottom spectrum shows peaks with variable fucosylation states that have been GalNAz modified.

FIGS. 11A-11B show that the One-Step DOS2 activation workflow results in antibodies labeled equivalently to a traditional three-step labeling workflow. FIG. 11A graphically represents quantification of sDIBO-AF488 labeling of antibodies after azide activation of the Fc glycans using either a traditional two-step antibody activation or the One-Step DOS2 workflow of the present disclosure. FIG. 11B shows an image of an SDS-PAGE visualization of AF488 labeling of antibody conjugates from FIG. 11A.

FIGS. 12A-12B show the One-Step DOS2 workflow results in antibodies activated with two GalNAz glycans. FIG. 12A shows the mass spectra of Endo S digested (top) and One-Step DOS2 azide activated anti-CD28 clone 28.2 antibody (bottom), indicating the One-Step DOS2 workflow results in antibodies substituted with two GalNAz moieties. FIG. 12B shows the absorbance spectra after labeling the azide-activated antibody with sDIBO-AF488.

FIGS. 13A-13B show the One-Step DOS2 workflow allows labeling across a variety of antibody isotypes. FIG. 13A shows a graph illustrating the degree of labeling by sDIBO-AF488 after preparation of indicated antibody isotypes using the One-Step DOS2 workflow. FIG. 13B shows images of SDS-PAGE visualization of antibody isotypes showing total protein (left) and AF488 labeled products (right). The identity of the antibodies used corresponding to each isotype is indicated in Table 2.

FIGS. 14A-14C show flow cytometry data indicating the One-Step DOS2 modified antibodies are functional in flow cytometry. Human PBMCs were stained anti-CD3 clone OKT3 conjugated to sDIBO-AF647 using the One-Step DOS2 workflow. CD3 OKT3 Alexa Fluor™ 647 (AF647) staining was mutually exclusive to CD19 eF450 staining and staining intensity varied with the amount of antibody used in staining: 1.0 g (FIG. 14A), 0.5 g (FIG. 14B), or 0.25 g (FIG. 14C).

### DETAILED DESCRIPTION

### Introduction

In general, IgG antibodies contain two N-linked glycans attached to specific conserved asparagine residues located in the antibody heavy chain Fc domain. These sugar chains, predominantly complex biantennary glycans with two terminal branches, are structurally quite homogeneous, and the terminal sequences of the glycan branches are highly consistent. Most of the antibody glycan branches terminate with galactose-N-acetylglucosamine (Gal-GlcNAc) or with N-acetylglucosamine (GlcNAc). Removal of the terminal Gal residue with β-galactosidase unmasks the majority of terminal GlcNAc labeling sites, and each N-linked glycan will contain, on average, 2 terminal GlcNAc residues per heavy chain (4 terminal GlcNAc per antibody). Endoglycosidases cleave the antibody glycan at the base or core GlcNAc, revealing one core GlcNAc labeling site per heavy chain (2 core GlcNAc per antibody). These terminal or core GlcNAc residues can subsequently be modified using a β-galactosyltransferase enzyme or a mutant thereof. The modification can be the addition of a reactive group or bioorthogonal group, such as an azide or a ketone group, or a biologically relevant molecule. In situations where a reactive or bioorthogonal group has been added to the antibody, a functionalized probe containing a corresponding reactive or bioorthogonal group can be used to attach a variety of compounds such as a detectable label, a therapeutic molecule, or a toxin. For example, the antibody can have an azide group attached to the terminal or core GlcNAc residues followed by reaction via copper-free click chemistry with a biologically or pharmaceutically active molecule that has been functionalized with a strained-ring alkyne.

The present disclosure provides methods for making activated antibodies and antibody conjugates that combine the necessary enzymatic reactions into a single step resulting in a faster and more efficient process for antibody conjugation. In one embodiment, the present disclosure provides methods for making activated antibodies or antibody conjugates with a degree of substitution (DOS), also known as degree of labeling (DOL), of about 4. In other embodiments, the present disclosure provides methods for making activated antibodies or antibody conjugates with a DOS of about 2. Also provided are activated antibodies and antibody conjugates made by the single step workflows of the present disclosure. Kits for making activated antibodies and antibody conjugates according to the single step workflows of the present disclosure are also provided.

The disclosed antibody conjugation methods are advantageous because antibody activation requires only a single incubation step. Specifically, the antibody, modified sugar substrate (also referred to herein as a modified sugar), and glycan-modifying enzymes (e.g., β-galactosidase and mutant β-1,4-galactosyltransferase, or mutant β-1,4-galactosyltransferase and endoglycosidase) are combined in a single reaction mixture and incubated together. A single activation step is desirable because it minimizes sample loss and increases reaction speed. In one embodiment, a β-galactosidase and a mutant β-1,4-galactosyltransferase are combined with a modified sugar substrate and incubated with an antibody for about 16 hours. In another embodiment, a mutant β-1,4-galactosyltransferase and an endoglycosidase are combined with a modified sugar substrate and incubated with an antibody for about 4 to 6 hours. These features contrast with existing site-specific antibody conjugation methods, which require a two-step activation process: incubation with β-galactosidase for at least 16 hours, followed by incubation with β-1,4-galactosyltransferase and modified sugar substrate for at least 30 hours, or incubation with an endoglycosidase for at least 16 hours, followed by incubation with a β-1,4-galactosyltransferase and modified sugar substrate for at least 30 hours. The one-step antibody activation reaction of the present disclosure was achieved by using a single buffer system compatible with the activity of both enzymes during antibody glycan modification. Each enzyme was titrated to determine the activity needed under the one-step conditions.

Without wishing to be bound by theory, previous site-specific antibody conjugation assays may not have been successful using a single enzymatic reaction for the antibody activation step because of the step-wise requirement of cleaving the glycan chains before adding the modified sugar substrate to the cleaved glycan chain, and the previously used reaction conditions for the different enzymes may not have been compatible when combined. In addition, continued presence of the endoglycosidase with the activated antibody has the potential to cleave the substituted sugar from the activated antibody.

### ABBREVIATIONS

Gal Galactose
GalNAz N-alpha-azidoacetylgalactosamine
GlcNAz N-alpha-azidoacetylglucosamine
GalNAc N-acetylgalactosamine
GlcNAc N-acetylglucosamine
NeuAc N-acetylneuraminic acid
GalKyne Alkyne-modified galactosamine
GalKetone Ketone-modified galactosamine
Gal-Cyclopropene Cyclopropene-modified galactosamine
Gal-Oxime Oxime-modified galactosamine
Gal-Nitrile oxide Nitrile oxide-modified galactosamine
GalVinyl Vinyl-modified galactosamine
GalVinylSulfo Vinylsulfo-modified galactosamine
GalVinylPhospho Vinylphospho-modified galactosamine
mAb monoclonal antibody
GalT β-1,4-galactosyltransferase
DOL Degree of labeling
DOS Degree of substitution
Endo Endoglycosidase

### SUMMARY OF TERMS

The section headings used herein are for organizational purposes only and are not to be construed as limiting the desired subject matter in any way. In the event that any of the incorporated literature contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Before describing the present teachings in detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise. Thus, for example, reference to “a ligand” includes a plurality of ligands and reference to “an antibody” includes a plurality of antibodies and the like.

Certain compounds disclosed herein can exist in unsolvated forms as well as solvated forms, including hydrated forms. In general, the solvated forms are equivalent to unsolvated forms and are encompassed within the scope of the present teachings.

Certain compounds disclosed herein may exist in multiple crystalline or amorphous forms. In general, all physical forms are equivalent for the uses contemplated by the present disclosure and are intended to be within the scope of the present disclosure.

Certain compounds disclosed herein possess asymmetric carbon atoms (optical centers) or double bonds; the racemates, diastereomers, geometric isomers and individual isomers are encompassed within the scope of the present teachings.

Where substituent groups are specified by their conventional chemical formulae, written from left to right, they equally encompass the chemically identical substituents, which would result from writing the structure from right to left, e.g., —CH₂O— is intended to also recite —OCH₂—.

Unless defined otherwise, all technical and scientific terms used herein have the same meaning as commonly understood by one of ordinary skill in the art to which this disclosure is related. The following terms are defined for purposes of the present disclosure.

The term “antibody” as used herein refers to an immunoglobulin molecule or immunologically active portion thereof, i.e., an antigen-binding portion. Examples of immunologically active portions of immunoglobulin molecules include immunoglobulin molecules or fragments thereof that comprise the F(ab) region and a sufficient portion of the Fc region to comprise the oligosaccharide linkage site, for example, the asparagine-GlcNAc linkage site (e.g., Asn 297). An antibody may be a polyclonal, monoclonal (mAb), recombinant (e.g., a chimeric or humanized), fully human, non-human (e.g., murine), or a single chain antibody. An antibody may have effector function and may fix complement, and may be coupled to a toxin or imaging agent. Antibodies may be endogenous or polyclonal wherein an animal is immunized to elicit a polyclonal antibody response or by recombinant methods resulting in monoclonal antibodies produced from hybridoma cells or other cell lines. It is understood that the term “antibody” as used herein includes within its scope any of the various classes or sub-classes of immunoglobulin derived from any of the animals conventionally used. An antibody may be, for example, an IgA, an IgD, an IgE, an IgG, an IgM, or an IgY.

The term “antibody fragments,” as used herein, refers to fragments of antibodies that retain the principal selective binding characteristics of the whole antibody, for example, a sufficient portion of the Fc region to comprise the oligosaccharide linkage site, for example, the asparagine-GlcNAc linkage site. Antibody fragments include Fc proteins.

The term “activated antibody,” as used herein, refers to an antibody that has undergone glycan modification with a chemical handle on heavy chain Fc domain GlcNAc residues using a mutant β-1,4-glycosyltransferase enzyme and a suitable modified sugar substrate. For example, an antibody that has a GalNAz attached to the terminal or core GlcNAc residue after reaction with a mutant β-1,4-glycosyltransferase enzyme is considered to be an activated antibody.

The term “azide-reactive,” as used herein, refers to a chemical moiety that selectively reacts with an azido group on another molecule to form a covalent chemical bond between the azido group and the azide reactive group. Examples of azide-reactive groups include, but are not limited to, phosphines, including, but not limited to, triarylphosphines, alkynes, including, but not limited to terminal alkynes, thiacycloalkynes, and strained-ring alkynes, such as cyclononynes described by Hodges et al, PCT Publication No. WO 2013/036748, cyclooctynes and difluorocyclooctynes described by Agard et al., J. Am. Chem. Soc., 126 (46):15046-15047 (2004), dibenzocyclooctynes described by Boons et al., PCT Publication No. WO 2009/067663 A1, and aza-dibenzocyclooctynes described by Debets et al., Chem. Comm., 46:97-99 (2010). The various difluorocyclooctynes and dibenzocyclooctynes described above are collectively referred to herein as cyclooctyne groups. “Azide-reactive” can also refer to a molecule that contains a chemical moiety that selectively reacts with an azido group.

The term “alkyne-reactive,” as used herein, refers to a chemical moiety that selectively reacts with an alkyne-modified group on another molecule to form a covalent chemical bond between the alkyne-modified group and the alkyne-reactive group. Examples of alkyne-reactive groups include, but are not limited to, azides. “Alkyne-reactive” can also refer to a molecule that contains a chemical moiety that selectively reacts with an alkyne group.

“Cyclooctyne” refers to a strained-ring alkyne containing a ring of eight carbon atoms connected by seven single bonds and one triple bond. Exemplary cyclooctynes include but are not limited to

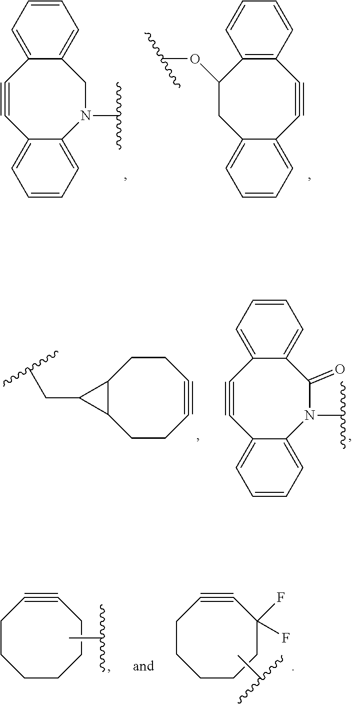

“Thiacycloalkyne” refers to a strained-ring alkyne that has a sulfur atom replacing a carbon atom and includes 3,3,6,6-tetramethylthiacycloheptyne (TMTH), TMTH-sulfoximine (TMTHSI) and the compounds described in de Almeida et al, Angew. Chem. Int. Ed. Engl. 51:2443 (2012).

The term, “chemical handle” as used herein refers to a specific functional group, such as an azide, an alkyne, including, but not limited to, a terminal alkyne, a strained-ring alkyne, a cyclooctyne, a cyclononyne, and a thiacycloalkyne, a phosphite, a phosphine, including, but not limited to a triarylphosphine, an oxime, a nitrile oxide and the like. A chemical handle is a moiety that is rarely found in naturally-occurring biomolecules and is chemically inert towards biomolecules (e.g., native cellular components), but when reacted with an azide-reactive or alkyne-reactive group the reaction can take place efficiently under biologically relevant conditions (e.g., cell culture conditions, such as in the absence of excess heat or harsh reactants). Chemical handles also include a Diels-Alder diene, a Diels-Alder dienophile, ketones, a straight or branched C₁-C₁₂ carbon chain bearing a carbonyl group, —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂(sulfonylhydrazide), —NR¹NR²(C═O)NR³NH₂ (carbazide), —NR¹NR²(C═S)NR³NH₂ (thiocarbazide), —ONH₂ (aminooxy), wherein each R¹, R², and R³ is independently H or alkyl having 1-6 carbons, vinyl groups, or thiols.

The terms “β-1,4-galactosyltransferase” and “GalT” are interchangeable.

The terms “endoglycosidase S2,” “Endo S2,” “endoglycosidase S49,” and “Endo S49” are interchangeable. Endo S49 is described in PCT Publication No. WO 2013/037824.

As used herein, the terms degree of labeling (DOL) and degree of substitution (DOS) are equivalent and interchangeable.

The term “click chemistry,” as used herein, refers to the Huisgen cycloaddition or the 1,3-dipolar cycloaddition between an azide and an alkyne to form a 1,2,4-triazole. Such chemical reactions can use, but are not limited to, simple heteroatomic organic reactants and are reliable, selective, stereospecific, and exothermic.

The term “cycloaddition” as used herein refers to a chemical reaction in which two or more π (pi)-electron systems (e.g., unsaturated molecules or unsaturated parts of the same molecule) combine to form a cyclic product in which there is a net reduction of the bond multiplicity. In a cycloaddition reaction, the π (pi) electrons are used to form new π (pi) bonds. The product of a cycloaddition is called an “adduct” or “cycloadduct”. Different types of cycloadditions are known in the art including, but not limited to, [3+2]cycloadditions and Diels-Alder reactions. [3+2]cycloadditions, which are also called 1,3-dipolar cycloadditions, occur between a 1,3-dipole and a dipolarophile and are typically used for the construction of five-membered heterocyclic rings. The term “[3+2]cycloaddition” also encompasses “copperless” [3+2]cycloadditions between azides and cyclooctynes and difluorocyclooctynes described by Agard et al., J. Am. Chem. Soc., 126 (46):15046-15047 (2004), dibenzocyclooctynes described by Boons et al., PCT Publication No. WO 2009/067663 A1, aza-dibenzocyclooctynes described by Debets et al., Chem. Comm., 46:97-99 (2010), cyclononynes described by Hodges et al, PCT Publication No. WO 2013/036748, and thiacycloalkynes described by de Almeida et al, Angew. Chem. Int. Ed. Engl. 51:2443 (2012).

The term “fluorophore” as used herein refers to a composition that is inherently fluorescent or demonstrates a change in fluorescence upon binding to a biological compound or metal ion, i.e., fluorogenic. Fluorophores may contain substituents that alter the solubility, spectral properties or physical properties of the fluorophore. Numerous fluorophores are known to those skilled in the art and include, but are not limited to coumarin, cyanine, benzofuran, quinoline, quinazolinone, indole, benzazole, borapolyazaindacene and xanthenes including fluorescein, rhodamine and rhodol, as well as other fluorophores described in Haugland, R., Molecular Probes Handbook of Fluorescent Probes and Research Chemicals (10^{th} edition, CD-ROM, September 2005).

The term “label,” as used herein, refers to a chemical moiety or protein that is directly or indirectly detectable (e.g. due to its spectral properties, conformation or activity) when attached to a target or compound, including reporter molecules, solid supports and biologically or pharmaceutically active molecules, and used in the present methods. The label can be directly detectable (e.g., fluorophore or radiolabel) or indirectly detectable (e.g., hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a colorimetric substrate (e.g., tetramethylbenzidine (TMB)) or a fluorogenic substrate such as Amplex™ Red reagent (Thermo Fisher Scientific, Waltham, MA) to detect the presence of HRP. Numerous labels are known by those of skill in the art and include, but are not limited to, particles, fluorophores, and other labels that are described in Haugland, R., Molecular Probes Handbook of Fluorescent Probes and Research Chemicals (10^{th} edition, CD-ROM, September 2005), supra.

The term “biologically or pharmaceutically active molecule” as used herein refers to a biological or a non-biological component that is covalently bonded to compound of the present disclosure. Such components include, but are not limited to, a reporter molecule, a radioisotope, a metal chelator, an amino acid, a peptide, a protein, a polysaccharide, a glycan, a nucleoside, a nucleotide, an oligonucleotide, a nucleic acid, a hapten, a psoralen, a drug, a cytotoxin, a prodrug, a diagnostic agent, a hormone, a lipid, a lipid nanoparticle, a liposome, a lipid assembly, a synthetic polymer, a polymeric microparticle, a biological cell, an antiviral agent, an antibacterial agent, an antifungal agent, a virus, a bead, a nanoparticle, a nanocrystal, a solid support, and combinations thereof. Examples of cytotoxins include, but are not limited to, camptothecins, staurosporin, doxorubicin, daunorubicin, colchicine, methotrexate, taxanes, calicheamycins, duocarmycins, maytansines and maytansinoids (i.e. maytansine derivatives), auristatins, tubulysin M, cryptophycin or pyrrolobenzodiazepines (PBDs). Examples of auristatins include dolastatin 10, auristatin F, monomethyl auristatin F (MMAF), auristatin E, monomethyl auristatin E (MMAE), auristatin PE, auristatin TP and auristatin AQ. Examples of maytansines and maytansinoids include mertansine and ansamitocin.

The term “reporter molecule” as used herein refers to any moiety capable of being attached to an activated antibody of the present disclosure, and detected either directly or indirectly. Reporter molecules include, without limitation, a chromophore, a fluorophore, a fluorescent protein, a phosphorescent dye, a tandem dye, a particle, a hapten, an enzyme, a radioisotope, a contrast agent, a magnetic resonance imaging agent or a mass label. Examples of a radioisotope include, but not limited to, ^{99m}Tc, ¹¹¹In, ¹⁸F, ¹⁴C, ⁶⁴Cu, ¹³¹I or ¹²³I, which may or may not be connected via a chelating moiety such as DTPA, DOTA, NOTA or HYNIC. Preferred reporter molecules include fluorophores, fluorescent proteins, haptens, and enzymes.

The term “solid support,” as used herein, refers to a material that is substantially insoluble in a selected solvent system, or which can be readily separated (e.g., by precipitation) from a selected solvent system in which it is soluble. The term solid support includes semi-solid supports. Solid supports useful in the present disclosure can include groups that are activated or capable of activation to allow selected species to be bound to the solid support. Solid supports can be present in a variety of forms, including a chip, wafer or well, onto which an individual, or more than one activated antibody or antibody conjugate of the present disclosure is bound such as a polymeric bead or particle. As used herein, a “solid support” includes, but is not limited to, an aerogel, a hydrogel, a resin, a silica gel, a bead, a biochip, a microfluidic chip, a silicon chip, a multi-well plate, a membrane, a polymeric membrane, a particle, a derivatized plastic film, a glass bead, cotton, a plastic bead, alumina gel, polysaccharide, poly(acrylate), polystyrene, poly(acrylamide), polyol, agarose, agar, cellulose, dextran, starch, heparin, glycogen, amylopectin, mannan, inulin, nitrocellulose, diazocellulose, polyvinylchloride, polypropylene, polyethylene, nylon, latex bead, a conducting metal, a nonconducting metal, glass, quantum dots, nanocrystals, nanoparticles, nanotubes, carbon nanotubes, graphene, magnetic bead, paramagnetic bead, superparamagnetic bead, and a magnetic support. In certain preferred embodiments, the solid support is chosen from a bead, a particle, a glass bead, a plastic bead, a latex bead, a quantum dot, a nanocrystal, a nanoparticle, a nanotube, a carbon nanotube, a graphene bead, a magnetic bead, a paramagnetic bead, a superparamagnetic bead, and a magnetic support.

The terms “workflow” and “method” are used interchangeably herein and refer to the method or process of steps to perform the one-step antibody activation and subsequent antibody conjugation reactions of the present disclosure.

### Improved Site-Specific Antibody Activation and Conjugation Workflows:

The present disclosure provides methods for making antibody conjugates that combine the necessary enzymatic reactions into a single step resulting in a faster and more efficient process for antibody conjugation. In one embodiment, the present disclosure provides methods for making activated antibodies or antibody conjugates with a degree of substitution (DOS), also known as degree of labeling (DOL), of about 4. In other embodiments, the present disclosure provides methods for making activated antibodies or antibody conjugates with a DOS of about 2. Also provided are activated antibodies and antibody conjugates made by the single step workflows of the present disclosure. Kits for making activated antibodies and antibody conjugates according to the single step workflows of the present disclosure are also provided.

The disclosed site-specific antibody activation and conjugation workflows improve upon currently available site-specific antibody conjugation methods because only a single activation incubation step is required. Specifically, the antibody, modified sugar substrate, and glycan modification enzymes (e.g., 3-galactosidase and mutant β-1,4-galactosyltransferase, or endoglycosidase and mutant β-1,4-galactosyltransferase) are combined in a single incubation step. A single activation step is desirable because it minimizes sample loss and improves reaction speed. These features contrast with currently available site-specific antibody conjugation methods, which require a two-step activation method: either incubation with β-galactosidase for at least 16 hours, followed by incubation with β-1,4-galactosyltransferase and modified sugar substrate for at least 30 hours, or incubation with an endoglycosidase for at least 16 hours, followed by incubation with β-1,4-galactosyltransferase and substrate for at least 30 hours. In one embodiment of the present disclosure, a one-step conjugation reaction is achieved by incubating the antibody with a reaction mixture containing both a β-galactosidase and a mutant β-1,4-galactosyltransferase and a modified sugar substrate for about 16 hours. In another embodiment of the present disclosure, a one-step conjugation reaction is achieved by incubating an antibody with a reaction mixture that contains both an endoglycosidase and a mutant β-1,4-galactosyltransferase and a modified sugar substrate for about 4 to 6 hours.

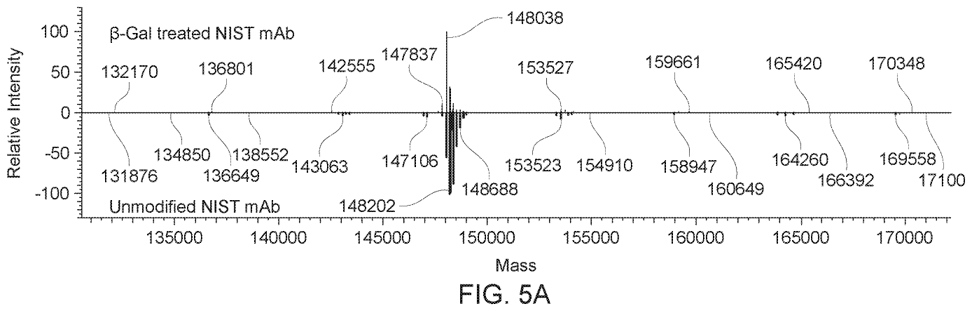

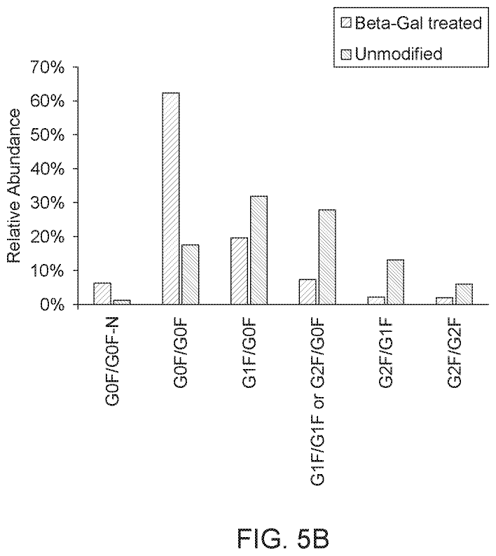

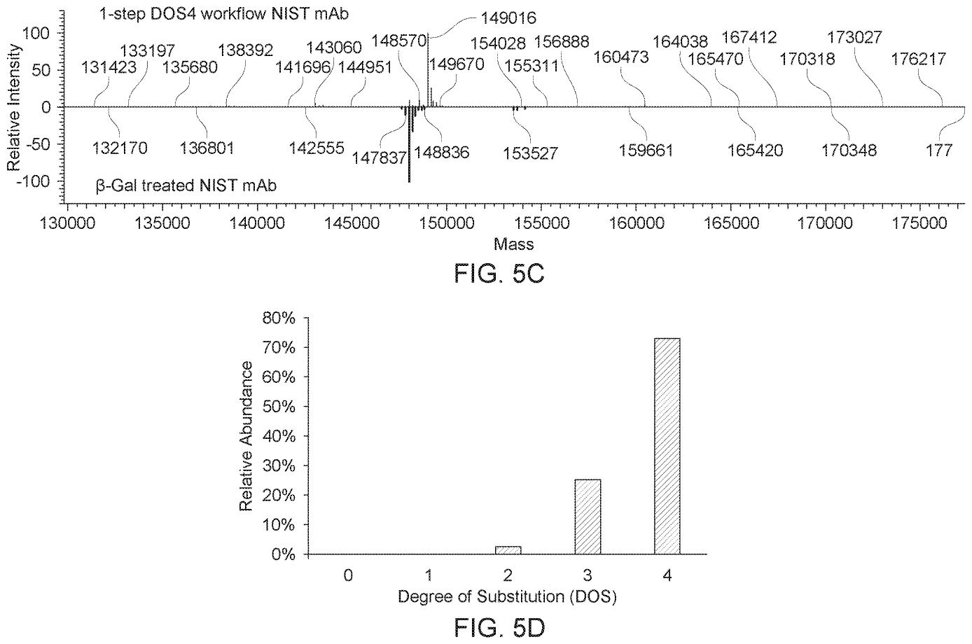

An existing single-step antibody glycosylation method utilizes fucosyltransferase enzymes to remove the fucose glycans from the GlcNAc residue on N297 before labeling due to the extent of crowding in the pocket filled by the double biantennary glycans (see, Yang et al, bioRxiv 2022.09.04.506510; doi: https://doi.org/10.1101/2022.09.04.506510). Unexpectedly, it was discovered that the one-step workflows of the present disclosure are able to produce modified antibody glycans regardless of the fucosylation state. Therefore, the one-step workflows of the present disclosure advantageously do not require removal of the fucose glycans before activation (see FIGS. 5A-5D).

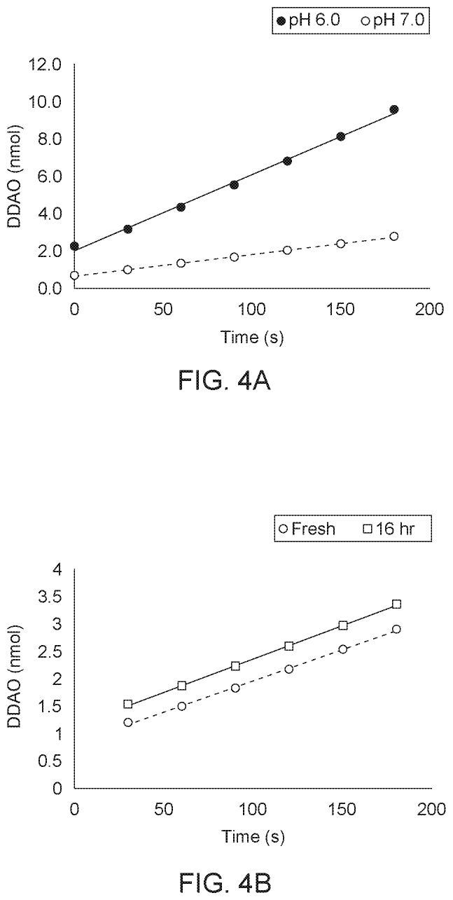

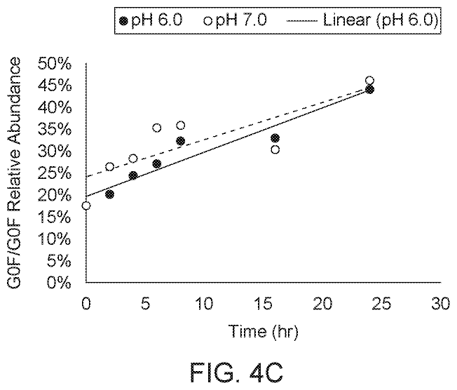

β-Galactosidase exhibits optimal enzymatic activity at pH 6.0 and suboptimal activity at pH 7.0, while the mutant β-1,4-galactosyltransferase (GalT) shows optimal activity at pH 7.0. Given the reduced activity of β-Galactosidase at pH 7.0, it was unexpected that the reactions of both enzymes could be carried out in a one-step reaction. Our measurements show that while the activity of β-Galactosidase on a small molecule substrate is substantially reduced at pH 7.0 relative to pH 6.0, activity on cleavage of an antibody glycan is not substantially reduced (see FIGS. 4A-4C).

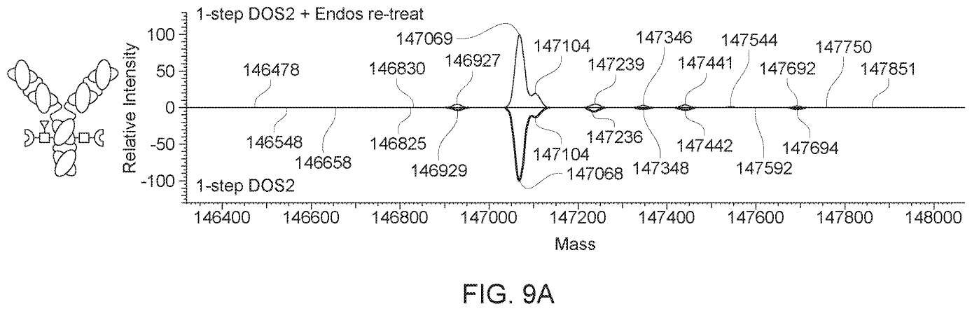

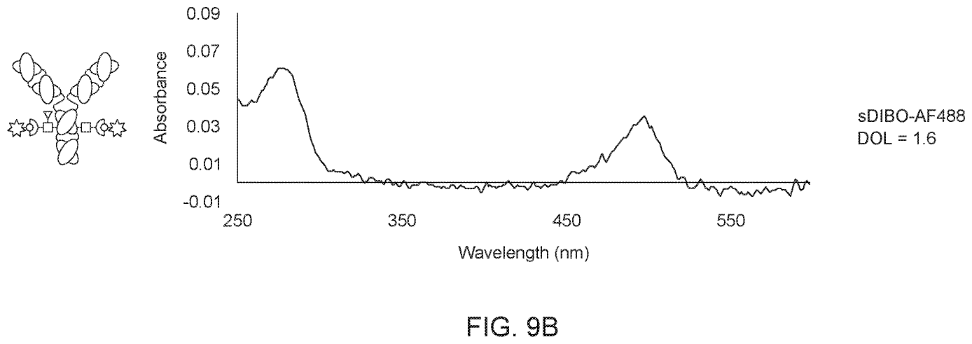

Endoglycosidase S (Endo S) cleaves the antibody glycan at the base or core GlcNAc. Previously it was thought that Endo S might be able to cleave the GalNAz attached to the core GlcNAc, which would preclude a one-step activation workflow as the Endo S must be removed before the galactosyltransferase reaction can be initiated. Surprisingly, our data shows that Endo S does not cleave the GalNAz attached to the antibody core GlcNAc making a one-step activation reaction possible (see FIGS. 9A-9B).

Provided herein are methods for activating an antibody using a single-step workflow. The activated antibodies according to the present disclosure can be conjugated to a variety of substances via a subsequent single-step reaction as described herein. The single-step workflows described herein allow for covalently attaching about 2 to about 4 chemical handles to antibodies without interfering with the antigen or Fc binding sites providing a streamlined workflow with lower cost and hands-on time in less than 24 hours. The attached chemical handles can be used as conjugation sites for reaction with biologically or pharmaceutically active molecules comprising a complementary chemical handle.

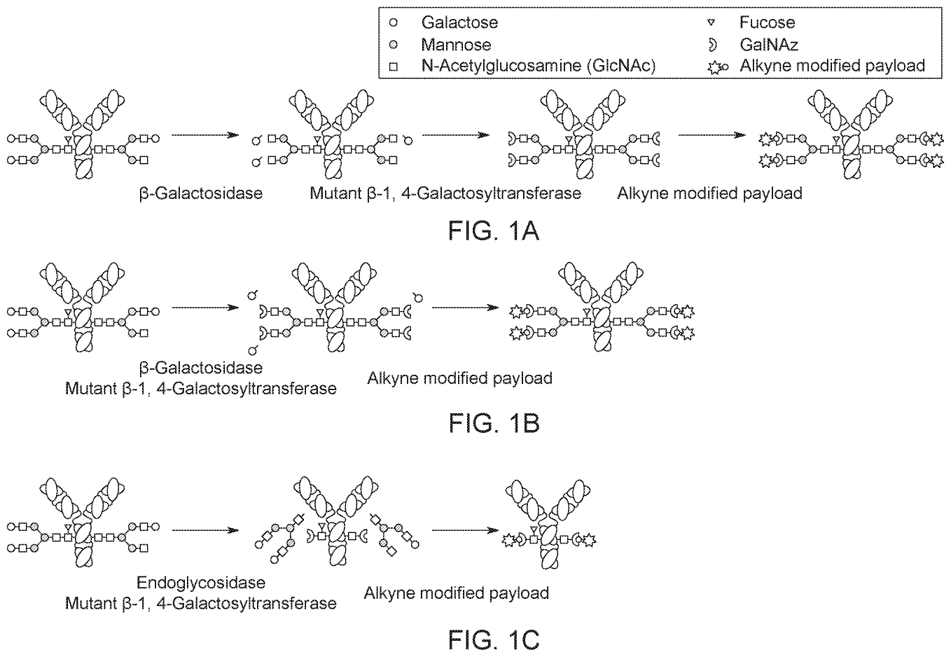

FIG. 1A shows an existing three-step antibody conjugation method which uses a two-step antibody activation workflow, in which the first step involves removal of terminal galactose residues from the heavy chain N-linked glycans of the antibody using β-galactosidase, exposing essentially all possible modifiable GlcNAc residues. In the second step, the free terminal GlcNAc residues are activated with azido tags by enzymatic attachment of GalNAz to the terminal GlcNAc residues using a β-1,4-galactosyltransferase enzyme, such as a mutant β-1,4-galactosyltransferase enzyme containing a Y289L mutation in the catalytic domain (“(Y289L)GalT”). In the third step, the azide residues are reacted with an alkyne-modified biologically or pharmaceutically active molecule resulting in a degree of labeling (DOL) of about 4 molecules per antibody.

In contrast, FIGS. 1B and 1C show exemplary two-step antibody conjugation methods which use a one-step antibody activation workflow according to embodiments of the present disclosure. In FIG. 1B, the first step involves combining the β-galactosidase and mutant β-1,4-galactosyltransferase enzymatic reactions in which the removal of the terminal galactose residues from the heavy chain N-linked glycans of the antibody and attachment of GalNAz to the free terminal GlcNAc residues occur in the same step. In the second step, the azide residues are reacted with an alkyne-modified biologically or pharmaceutically active molecule resulting in a degree of labeling (DOL) of about 4 molecules per antibody. In FIG. 1C, the first step involves combining the mutant β-1,4-galactosyltransferase and endoglycosidase enzymatic reactions in which the core GlcNAc residues of the heavy chain N-linked glycans of the antibody are exposed and attachment of GalNAz to the free core GlcNAc residues occur in the same step. In the second step, the azide residues are reacted with an alkyne-modified biologically or pharmaceutically active molecule resulting in a degree of labeling (DOL) of about 2 molecules per antibody.

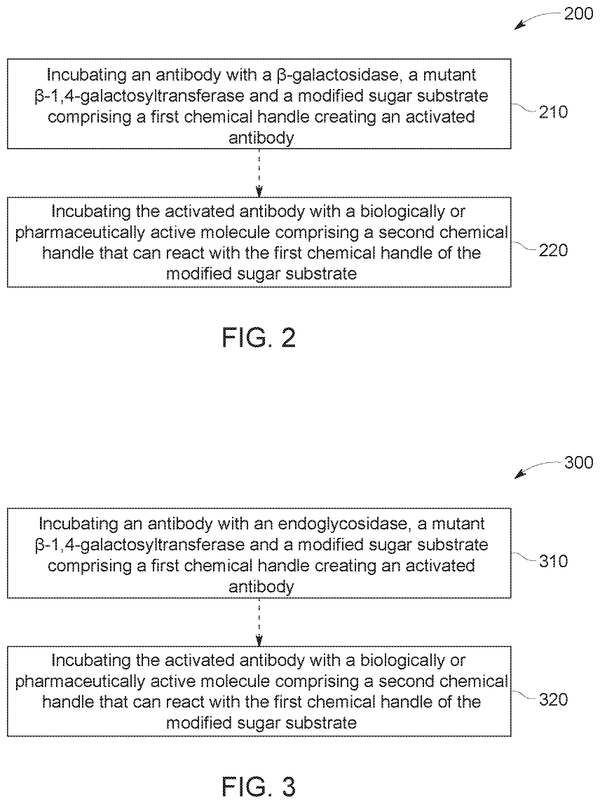

FIG. 2 shows a flow-chart of an exemplary two-step method (200) of conjugating an antibody with a one-step antibody activation workflow according to the present disclosure. Step 210 is incubating the antibody with a β-galactosidase, a mutant β-1,4-galactosyltransferase and a modified sugar substrate comprising a first chemical handle creating an activated antibody. Step 210 is a simultaneous incubation of the β-galactosidase, mutant β-1,4-galactosyltransferase and modified sugar substrate comprising a first chemical handle in a common buffer. Step 220 is incubating the activated antibody with a biologically or pharmaceutically active molecule comprising a second chemical handle that can react with the first chemical handle of the modified sugar substrate. The dashed arrow between Step 210 and Step 220 indicates that the workflow can terminate with Step 210, or that Step 220 can follow Step 210. In certain embodiments, only Step 210 is performed to provide an activated antibody that can be stored for further conjugation at a later time. In certain other embodiments, Step 220 is performed immediately after Step 210. In certain embodiments, Step 220 follows Step 210 after a period of time. In certain embodiments, the period of time separating Step 210 from Step 220 can be minutes, hours, or days.

FIG. 3 shows a flow-chart of an exemplary two-step method (300) of conjugating an antibody with a one-step antibody activation workflow according to the present disclosure. Step 310 is incubating the antibody with an endoglycosidase, a mutant β-1,4-galactosyltransferase and a modified sugar substrate comprising a first chemical handle creating an activated antibody. Step 310 is a simultaneous incubation of the endoglycosidase, mutant β-1,4-galactosyltransferase and modified sugar substrate comprising a first chemical handle in a common buffer. Step 320 is incubating the activated antibody with a biologically or pharmaceutically active molecule comprising a second chemical handle that can react with the first chemical handle of the modified sugar substrate. The dashed arrow between Step 310 and Step 320 indicates that the workflow can terminate with Step 310, or that Step 320 can follow Step 310. In certain embodiments, only Step 310 is performed to provide an activated antibody that can be stored for further conjugation at a later time. In certain other embodiments, Step 320 is performed immediately after Step 310. In certain embodiments, Step 320 follows Step 310 after a period of time. In certain other embodiments, the period of time separating Step 310 from Step 320 can be minutes, hours, or days.

In certain embodiments, the antibody comprises a terminal or core GlcNAc residue. In certain embodiments, the terminal or core GlcNAc residue may be obtained by incubating the antibody with an enzyme such as a β-galactosidase or an endoglycosidase. In certain embodiments, the antibody is an IgA, an IgE, an IgD, an IgG, an IgM, or an IgY. In certain embodiments, the terminal or core GlcNAc residues are present on the Fc region of the antibody. In certain embodiments, the terminal or core GlcNAc residues are attached to an asparagine residue in the Fc region of the antibody. In certain embodiments, the asparagine in the Fc region is at amino acid position 297 (Asn297).

The antibody may be present in the methods of the present disclosure at a concentration selected to provide a desired degree of conjugation, reaction efficiency, and/or product yield. In some embodiments, the antibody is present at a concentration of about 0.5 mg/mL to about 10 mg/mL. In certain embodiments, the antibody concentration is selected from about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, about 3 mg/mL, about 3.25 mg/mL, about 3.5 mg/mL, about 3.75 mg/mL, about 4 mg/mL, about 4.25 mg/mL, about 4.5 mg/mL, about 4.75 mg/mL, about 5 mg/mL, about 5.25 mg/mL, about 5.5 mg/mL, about 5.75 mg/mL, about 6 mg/mL, about 6.25 mg/mL, about 6.5 mg/mL, about 6.75 mg/mL, about 7 mg/mL, about 7.25 mg/mL, about 7.5 mg/mL, about 7.75 mg/mL, about 8 mg/mL, about 8.25 mg/mL, about 8.5 mg/mL, about 8.75 mg/mL, about 9 mg/mL, about 9.25 mg/mL, about 9.5 mg/mL, about 9.75 mg/mL, and about 10 mg/mL. In certain embodiments, the antibody concentration is defined by any two of the foregoing values as endpoints. In particular embodiments, the antibody concentration is about 0.5 mg/mL to about 1 mg/mL, about 1 mg/mL to about 5 mg/mL, or about 5 mg/mL to about 10 mg/mL. In a particular embodiment, the antibody concentration is about 1 mg/mL.

In certain embodiments, the first chemical handle of the modified sugar substrate comprises an azide group, an alkyne group, a strained-ring alkyne group, a cycloalkene group, a ketone group, an oxime group, a nitrile oxide group, or a vinyl group. In certain embodiments, the modified sugar substrate is chosen from UDP-GalNAz, UDP-GalKyne, UDP-Gal-Cyclopropene, UDP-GalKetone, UDP-Gal-Oxime, UDP-Gal-Nitrile oxide, UDP-GalVinyl, UDP-GalVinylSulfo, and UDP-GalVinylPhospho. In certain embodiments, the modified sugar substrate is chosen from:

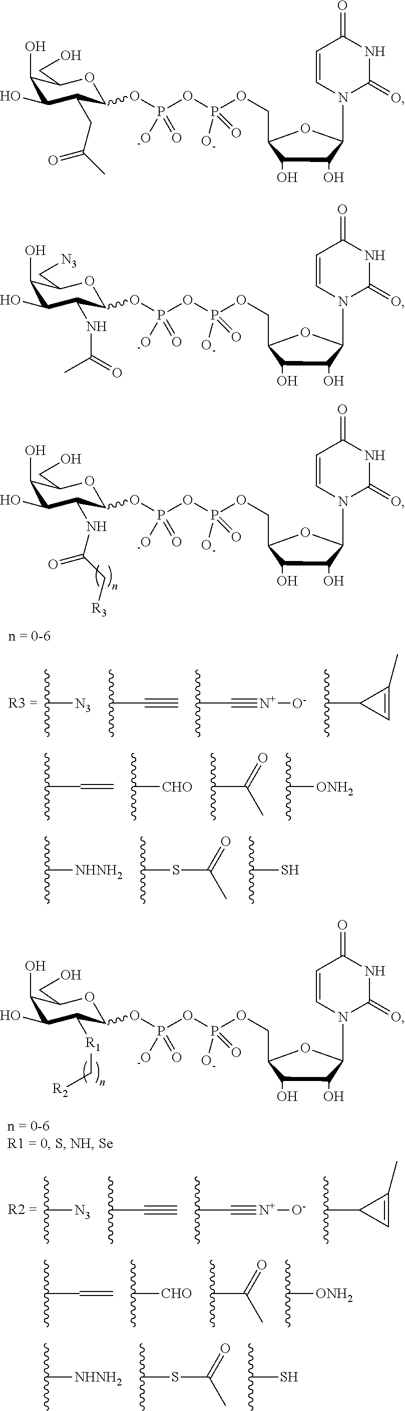

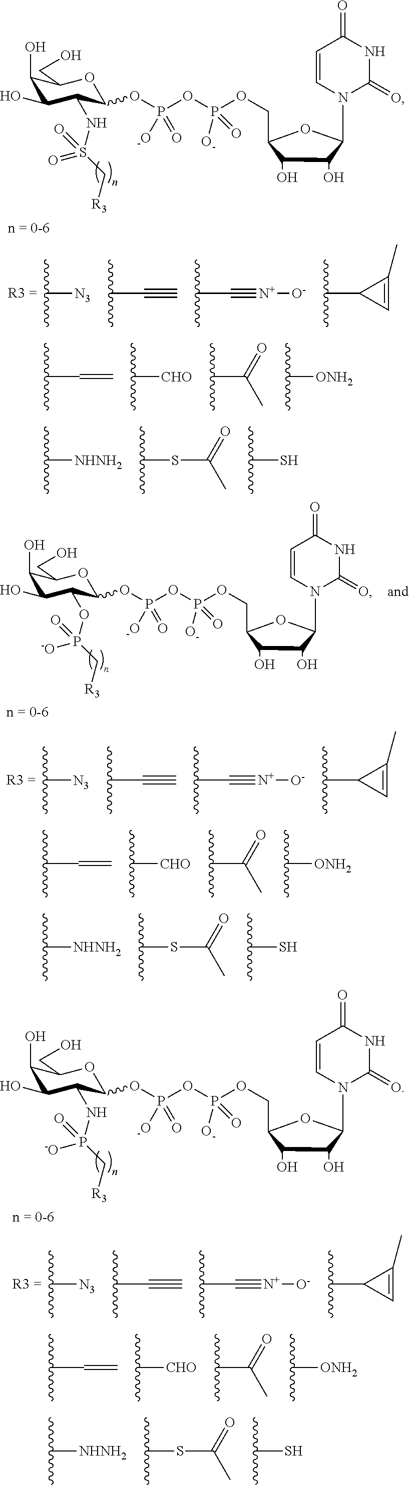

In certain preferred embodiments, the modified sugar substrate is UDP-GalNAz.

The modified sugar substrate may be present in the reaction mixture at a concentration selected to provide a desired level of product formation. In certain embodiments, the modified sugar substrate is present at a concentration of about 0.5 mM to about 5 mM. In certain embodiments, the concentration of the modified sugar substrate is about 0.5 mM, about 0.75 mM, about 1 mM, about 1.5 mM, about 1.75 mM, about 2 mM, about 2.5 mM, about 3 mM, about 3.5 mM, about 4 mM, about 4.5 mM, or about 5 mM. In certain embodiments, the modified sugar substrate concentration is defined by any two of the foregoing values as endpoints. In particular embodiments, the modified sugar substrate concentration is about 0.5 mM to about 1 mM, about 1 mM to about 2.5 mM, or about 2.5 mM to about 5 mM. In a particular embodiment, the modified sugar substrate concentration is about 2 mM.

In certain embodiments, the second chemical handle attached to the biologically or pharmaceutically active molecule comprises an alkyne-reactive group, an azide-reactive group, a Diels-Alder diene, a Diels-Alder dienophile, a straight chain or branched C₁-C₁₂ carbon chain bearing a carbonyl group, a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂(sulfonylhydrazide), —NR¹NR²(C═O)NR³NH₂ (carbazide), —NR¹NR²(C═S)NR³NH₂ (thiocarbazide), —ONH₂ (aminooxy), wherein each R¹, R², and R³ is independently H or alkyl having 1-6 carbons, or a thiol. In certain preferred embodiments, the second chemical handle comprises a strained ring alkyne.

In certain embodiments, the first chemical handle of the modified sugar substrate comprises an azide group, and the second chemical handle of the biologically or pharmaceutically active molecule comprises a terminal triarylphosphine, terminal alkyne, or strained-ring alkyne group. In certain embodiments, the first chemical handle of the modified sugar substrate comprises a terminal triarylphosphine, terminal alkyne or activated alkyne group, and the second chemical handle of the biologically or pharmaceutically active molecule comprises an azide group. In certain embodiments, the strained-ring alkyne comprises a cyclooctyne, a cyclononyne, or a thiacycloalkyne. In certain preferred embodiments, the first chemical handle of the modified sugar substrate is an azide and the second chemical handle of the biologically or pharmaceutically active molecule is a strained-ring alkyne.

In certain embodiments, the first chemical handle of the modified sugar substrate comprises an oxime group or a nitrile oxide group, and the second chemical handle of the biologically or pharmaceutically active molecule comprises a strained-ring alkyne group. In certain embodiments, the first chemical handle of the modified sugar substrate comprises a strained-ring alkyne group and the second chemical handle of the biologically or pharmaceutically active molecule comprises an oxime group or a nitrile oxide group. In certain embodiments, the strained-ring alkyne group is a cyclooctyne group.

In certain embodiments, the first chemical handle of the modified sugar substrate comprises a Diels-Alder diene and the second chemical handle of the biologically or pharmaceutically active molecule comprises a Diels-Alder dienophile. In certain embodiments, the first chemical handle of the modified sugar substrate comprises a Diels-Alder dienophile and the second chemical handle of the biologically or pharmaceutically active molecule comprises a Diels-Alder diene.

In certain embodiments, the first chemical handle of the modified sugar substrate comprises a straight chain or branched C₁-C₁₂ carbon chain bearing a carbonyl group, and the second chemical handle of the biologically or pharmaceutically active molecule comprises a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR³NH₂ (carbazide), —NR¹NR²(C═S)NR³NH₂ (thiocarbazide), or —ONH₂ (aminooxy), wherein each R¹, R², and R³ is independently H or alkyl having 1-6 carbons.

In certain embodiments, the first chemical handle of the modified sugar substrate comprises a cycloalkene and the second chemical handle of the biologically or pharmaceutically active molecule comprises a tetrazine. In certain embodiments, the first chemical handle of the modified sugar substrate comprises a tetrazine and the second chemical handle of the biologically or pharmaceutically active molecule comprises a cycloalkene.

In certain embodiments, the first chemical handle of the modified sugar substrate comprises a vinyl, a vinylsulfo, or a vinylphospho group and the second chemical handle of the biologically or pharmaceutically active molecule comprises a thiol.

The β-galactosidase enzyme may be present in the reaction mixture at a concentration selected to remove terminal galactose residues from the heavy chain N-linked glycans of the antibody, exposing essentially all possible modifiable GlcNAc residues, optionally with a fucose residue attached. In some embodiments, the β-galactosidase enzyme is present at a concentration of about 0.005 mg/mL to about 1 mg/mL. In certain embodiments, the β-galactosidase enzyme concentration is selected from about 0.005 mg/mL, about 0.01 mg/mL, about 0.015 mg/mL, about 0.02 mg/mL, about 0.05 mg/mL, about 0.075 mg/mL, about 0.1 mg/mL, about 0.25 mg/mL, about 0.5 mg/mL, about 0.75 mg/mL, and about 1 mg/mL. In certain embodiments, the β-galactosidase enzyme concentration is defined by any two of the foregoing values as endpoints. In particular embodiments, the β-galactosidase enzyme concentration is about 0.005 mg/mL to about 0.05 mg/mL, about 0.01 mg/mL to about 0.1 mg/mL, about 0.05 mg/mL to about 0.5 mg/mL, or about 0.1 mg/mL to about 1 mg/mL. In a particular embodiment, the β-galactosidase enzyme concentration is about 0.015 mg/mL.

The β-1,4-galactosyltransferase enzyme may be present in the reaction mixture at a concentration selected to catalyze the attachment of GalNAz to the terminal GlcNAc residues. In certain embodiments, the β-1,4-galactosyltransferase enzyme concentration is selected from about 0.005 mg/mL, about 0.01 mg/mL, about 0.015 mg/mL, about 0.02 mg/mL, about 0.05 mg/mL, about 0.075 mg/mL, about 0.1 mg/mL, about 0.25 mg/mL, about 0.5 mg/mL, about 0.75 mg/mL, and about 1 mg/mL. In certain embodiments, the β-1,4-galactosyltransferase enzyme concentration is defined by any two of the foregoing values as endpoints. In particular embodiments, the β-1,4-galactosyltransferase enzyme concentration is about 0.005 mg/mL to about 0.05 mg/mL, about 0.01 mg/mL to about 0.1 mg/mL, about 0.05 mg/mL to about 0.5 mg/mL, or about 0.1 mg/mL to about 1 mg/mL. In a particular embodiment, the β-1,4-galactosyltransferase enzyme concentration is about 0.015 mg/mL.

In certain embodiments, the β-1,4-galactosyltransferase enzyme is a mutant β-1,4-galactosyltransferase enzyme. In certain embodiments, the mutant β-1,4-galactosyltransferase enzyme comprises a mutation in the catalytic domain. β-1,4-galactosyltransferase enzymes with mutations in the catalytic domain, along with methods of preparation, are disclosed in, for example, PCT Publication No. WO 2004/063344, Bojarovi et al., Glycobiology 19:509 (2009), and Qasba et al., Glycobiology 12:691 (2002). The mutant β-1,4-galactosyltransferase domains may be included within a full-length β-1,4-galactosyltransferase enzyme, or in a recombinant molecule containing the catalytic domains. In certain embodiments, the mutation can be at residue Tyr-289, Tyr-284, or Arg-228. In certain embodiments, the β-1,4-galactosyltransferase is a Y289L, Y289N, Y289I, Y289F, Y289M, Y289V, Y289G, or Y289A mutant. In certain preferred embodiments, the β-1,4-galactosyltransferase is a Y289L mutant, also referred to herein as a (Y289L)GalT mutant.

In some embodiments, the reaction mixture comprises MnCl₂ as a cofactor in an amount of about 1 mM to about 50 mM. In certain embodiments, MnCl₂ is present in an amount selected from about 1 mM, about 2 mM, about 3 mM, about 4 mM, about 5 mM, about 6 mM, about 7 mM, about 8 mM, about 9 mM, about 10 mM, about 12 mM, about 15 mM, about 20 mM, about 25 mM, about 30 mM, about 35 mM, about 40 mM, about 45 mM, and about 50 mM. In certain embodiments, MnCl₂ is present in an amount defined by any two of the foregoing values as endpoints. Illustrative ranges include about 1 mM to about 40 mM, about 1 mM to about 30 mM, about 1 mM to about 20 mM, about 2 mM to about 20 mM, about 2 mM to about 15 mM, about 5 mM to about 20 mM, about 5 mM to about 15 mM, about 8 mM to about 12 mM, and about 9 mM to about 11 mM. In exemplary embodiments, MnCl₂ is present at about 10 mM.

The endoglycosidase enzyme may be present in the reaction mixture at a concentration selected to cleave the glycosidic bond within the chitobiose core of an Fc-associated N-glycan of an immunoglobulin, thereby leaving a residual N-acetylglucosamine residue, optionally with a fucose residue, attached. In some embodiments, the enzyme is present at a concentration of about 0.5 U/μL to about 3 U/μL, including any value or subrange therebetween. For example, the enzyme concentration may be about 0.5 U/μL, about 0.75 U/μL, about 1 U/μL, about 1.5 U/μL, about 2 U/μL, about 2.5 U/μL, or about 3 U/μL. In certain embodiments, the enzyme is present at a concentration of about 0.5 U/μL to about 1 U/μL, about 1 U/μL to about 2 U/μL, or about 2 U/μL to about 3 U/μL. In a particular embodiment, the enzyme is present at a concentration of about 1 U/μL.

In certain embodiments, the endoglycosidase (Endo) is chosen from Endo S, Endo S2, Endo A, Endo F, Endo M, Endo D and Endo H, or a combination thereof, the selection of which depends on the nature of the glycan. In certain embodiments, the endoglycosidase is Endo S. In preferred embodiments, the endoglycosidase is Endo S2.

The one-step activation workflows of the present disclosure result in activated antibodies that can be further conjugated to a variety of compounds via the chemical handle, including biologically or pharmaceutically active molecules. In certain embodiments, the activated antibody can be conjugated to a reporter molecule. In certain embodiments, the reporter molecule is chosen from a fluorescent dye, an enzyme, a radioisotope, a metal chelator, or a detectable substrate. In certain embodiments, the activated antibody can be conjugated to an amino acid, a peptide, a protein, a polysaccharide, a glycan, a nucleoside, a nucleotide, an oligonucleotide, a nucleic acid, a hapten, a psoralen, a drug, a cytotoxin, a prodrug, a diagnostic agent, a hormone, a lipid, a lipid nanoparticle, a lipid assembly, a synthetic polymer, a polymeric microparticle, a biological cell, an antiviral agent, an antibacterial agent, an antifungal agent, a virus or a combination thereof. In certain embodiments, the activated antibody can be conjugated to a solid support.

In certain embodiments, the labels or tags can be, for example, a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, anthraquinones, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the biologically or pharmaceutically active molecule may be DNA or a protein or polypeptide possessing a desired biological activity. Such proteins may include, for example, a toxin such as abrin, ricin A, Pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. In certain aspects, an antibody can be conjugated to a second antibody to form an antibody heteroconjugate as described by Segal in U.S. Pat. No. 4,676,980, for example, or a bispecific antibody.

The labels or tags can also, for example, be detectable labels used, for example, for diagnostic or research purposes, or tags used for binding of the antibody to other biomolecules or reagents (such as, for example, biotin/avidin binding, or binding of the antibody to microarray chips, beads, or plates). Labels or tags can also include any directly or indirectly detectable reporter molecule known by one skilled in the art that can be attached to an activated antibody of the present disclosure. Reporter molecules include, without limitation, a chromophore, a fluorophore, a fluorescent protein, a phosphorescent dye, a tandem dye, a particle, a hapten, an enzyme and a radioisotope. Preferred reporter molecules include fluorophores, fluorescent proteins, haptens, and enzymes.

A fluorophore is any chemical moiety that exhibits an absorption maximum beyond 280 nm, and when covalently attached to a labeling reagent retains its spectral properties. Fluorophores can include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1, 3-diazole (NBD), a cyanine (including any corresponding compounds in U.S. Pat. Nos. 6,977,305 and 6,974,873), a carbocyanine (including any corresponding compounds in U.S. Pat. Nos. 6,664,047; 6,974,873 and 6,977,305; 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; PCT Publication Nos. WO 02/26891, WO 97/40104, WO 99/51702, WO 01/21624; and EP Publication No. EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and 6,716,979), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045), or a rhodamine (including any corresponding compounds in U.S. Pat. Nos. 5,798,276; 5,846,737; and 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position. Xanthenes can also include analogs or derivatives of a xanthene including, but not limited to, fluorescein, eosins, rhodamines, including rhodamines substituted with a Group 13, Group 14 or Group 15 atom at position 10 of the xanthene ring, including, but not limited to Boron-rhodamines (B-rhodamines), Carbon-rhodamines (C-rhodamines), Silicon-rhodamines (Si-rhodamines), Germanium-rhodamines (Ge-rhodamines), Tin-rhodamines (Sn-rhodamines), Nitrogen-rhodamines (N-rhodamines), Phosphorous-rhodamines (P-rhodamines), Sulfur-rhodamines (S-rhodamines), Selenium-rhodamines (Se-rhodamines), and Tellurium-rhodamines (Te-rhodamines) [Deng and Xu, Chin. Chem. Lett. 30:1667-1681 (2019)]. Any commercial rhodamine dye and their analog in this category are included, for example Alexa Fluor™ dyes (Thermo Fisher Scientific, Waltham, MA), DyLight™ dyes (Thermo Fisher Scientific, Waltham, MA), ATTO dyes (ATTO-TEC GmbH, Siegen, Germany) and CF™ dyes (Biotium, Inc., Hayward, CA). Also included are hybrid rhodamine dyes and asymmetrical rhodamine dyes.

The choice of the fluorophore attached to the labeling reagent will determine the absorption and fluorescence emission properties of the labeling reagent and immuno-labeled complex. Physical properties of a fluorophore label include spectral characteristics (absorption, emission and stokes shift), fluorescence intensity, lifetime, polarization and photo-bleaching rate, all of which can be used to distinguish one fluorophore from another. Fluorophores also include fluorescently-labeled oligonucleotide nanostructures, such as those disclosed in U.S. Pat. No. 11,674,901. Any commercial fluorescently-labeled oligonucleotide nanostructures are included, for example NovaFluor™ dyes (Thermo Fisher Scientific, Waltham, MA).

In addition to fluorophores, enzymes also find use as labels for the detection reagents. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. However, fluorophores are preferred because they do not require additional assay steps and thus reduce the overall time required to complete an assay. The enzyme substrate is selected to yield the preferred measurable product, e.g. colorimetric, fluorescent or chemiluminescence. Such substrates are extensively used in the art, many of which are described in the MOLECULAR PROBES HANDBOOK, supra.

A preferred colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex™ Red reagent and its variants (U.S. Pat. No. 4,384,042), Amplex™ UltraRed and its variants in (PCT Publication No. WO 05/042504) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another preferred colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Glycosidases, in particular D-galactosidase, D-glucuronidase and D-glucosidase, are additional suitable enzymes. Appropriate colorimetric substrates include, but are not limited to, 5-bromo-4-chloro-3-indolyl β-D-galactopyranoside (X-gal) and similar indolyl galactosides, glucosides, and glucuronides, o-nitrophenyl β-D-galactopyranoside (ONPG) and p-nitrophenyl β-D-galactopyranoside. Preferred fluorogenic substrates include resorufin D-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl β-D-galactopyranoside, carboxyumbelliferyl β-D-galactopyranoside and fluorinated coumarin β-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In addition to enzymes, haptens such as biotin are also preferred labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

The activated antibodies described herein can be conjugated to a solid support. Solid supports can be present in a variety of forms, including a polymeric bead or particle. The solid support can include, but is not limited to, a resin, a silica gel, a bead, a biochip, a microfluidic chip, a silicon chip, a multi-well plate, a membrane, a polymeric membrane, a particle, a glass bead, a plastic bead, alumina gel, polysaccharide, poly(acrylate), polystyrene, poly(acrylamide), polyol, dextran, a latex bead, quantum dots, nanocrystals, nanoparticles, nanotubes, carbon nanotubes, graphene, magnetic bead, paramagnetic bead, superparamagnetic bead, and a magnetic support. The solid support can be chosen from a bead, a particle, a glass bead, a plastic bead, a latex bead, a quantum dot, a nanocrystal, a nanoparticle, a nanotube, a carbon nanotube, a graphene bead, a magnetic bead, a paramagnetic bead, a superparamagnetic bead, and a magnetic support.

Various buffers can be used in the methods described herein, including inorganic and organic buffers. In certain embodiments the organic buffer is a zwitterionic buffer. By way of example only, buffers that can be used in the methods described herein include succinate, citrate, maleate, cacodylate, N-(2-Acetamido)iminodiacetic acid (ADA), 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxypropanesulfonic acid (MOPSO), N,N-bis-(hydroxyethyl)-2-aminoethanesulfonic acid (BES), 3-(N-morpholino)-propanesulfonic acid (MOPS), N-tris-(hydroxymethyl)-2-ethanesulfonic acid (TES), N-2-hydroxyethyl-piperazine-N-2-ethanesulfonic acid (HEPES), 3-(N-tris-(hydroxymethyl) methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)piperazine-N′-(2-hydroxypropanesulfonic acid) (HEPPSO), 4-(2-Hydroxyethyl)-1-piperazinepropanesulfonic acid (EPPS), N-[Tris(hydroxymethyl)methyl]glycine (Tricine), N,N-Bis(2-hydroxyethyl)glycine (Bicine), (2-Hydroxy-1,1-bis(hydroxymethyl)ethyl)amino]-1-propanesulfonic acid (TAPS), N-(1,1-Dimethyl-2-hydroxyethyl)-3-amino-2-hydroxypropanesulfonic acid (AMPSO), tris (hydroxy methyl) amino-methane (Tris), Tris-Acetate-EDTA (TAE), glycine, bis[2-hydroxyethyl]iminotris[hydroxymethyl]methane (BisTris), or combinations thereof. In certain embodiments, the buffer comprises Tris. In certain embodiments, the buffer comprises MES. In certain embodiments, the buffer comprises PIPES. In certain embodiments, the buffer comprises Bis-Tris. In certain embodiments, the buffer comprises MOPS. In certain embodiments, the buffer comprises TES. In certain embodiments, the buffer comprises HEPES. In certain embodiments, the buffer comprises Tricine. In certain embodiments, the buffer comprises Bicine. In certain embodiments, the buffer comprises one or more of MES, PIPES, Bis-Tris, MOPS, TES, HEPES, Tricine, and Bicine. In certain embodiments, the buffer can also include a chelator, such as ethylenediaminetetraacetic acid (EDTA), or amino acids. In certain embodiments, the chelator is present at a concentration of about 0.1 mM to about 5 mM, including any value or subrange therebetween. For example, the chelator may be present at a concentration of about 0.1 mM, about 0.25 mM, about 0.5 mM, about 1 mM, about 2 mM, about 2.5 mM, or about 5 mM. In certain embodiments, the chelator is present at a concentration of about 0.1 mM to about 0.5 mM, about 0.5 mM to about 1 mM, about 1 mM to about 2.5 mM, or about 2.5 mM to about 5 mM. In a particular embodiment, the chelator comprises EDTA at a concentration of about 0.1 mM.

In certain embodiments, the concentration of such buffers used in the methods described herein is from about 0.1 mM to 1 M, including any value or subrange therebetween. For example, the buffer concentration may be about 0.1 mM, 0.5 mM, 1 mM, 5 mM, 10 mM, 25 mM, 50 mM, 75 mM, 100 mM, 125 mM, 150 mM, 200 mM, 250 mM, 500 mM, 750 mM, or 1 M. In certain embodiments, the buffer concentration is from about 10 mM to about 1 M. In certain embodiments, the buffer concentration is from about 20 mM to about 500 mM. In certain embodiments, the buffer concentration is from about 50 mM to about 300 mM. In certain embodiments, the buffer concentration is from about 0.1 mM to about 50 mM. In certain embodiments, the buffer concentration is from about 0.5 mM to about 20 mM. In further embodiments, the buffer concentration is from about 1 mM to about 100 mM, from about 5 mM to about 200 mM, from about 10 mM to about 100 mM, from about 25 mM to about 75 mM, or from about 40 mM to about 60 mM. In certain embodiments, the buffer concentration is about 10 mM, about 20 mM, about 25 mM, about 50 mM, about 75 mM, about 100 mM, about 200 mM, about 300 mM, about 500 mM, or about 1 M. In a particular embodiment, the buffer concentration is about 50 mM.

In certain embodiments, buffers used in the methods described herein have a pH between 5 and 9 at ambient temperature. In certain embodiments, the buffer has a pH of about 5.5 to about 8.5, about 6 to about 8.5, about 6 to about 8, about 6 to about 7.5, about 6 to about 7, or about 6.5 to about 7.5 at ambient temperature. In certain embodiments, the buffer has a pH of about 6.8 to about 7.2 at ambient temperature. In certain embodiments, the buffer has a pH of about 7.0 at ambient temperature. In certain embodiments the buffer has a pH between 5 and 9 at 25 °C. In certain embodiments, the buffer has a pH of about 5.5 to about 8.5, about 6 to about 8.5, about 6 to about 8, about 6 to about 7.5, about 6 to about 7, or about 6.5 to about 7.5 at 25 °C. In certain embodiments, the buffer has a pH of about 6.8 to about 7.2 at 25 °C. In certain embodiments, the buffer has a pH of about 7.0 at 25 °C. In further embodiments, the buffer may have a pH of about 5, about 5.5, about 6, about 6.5, about 6.8, about 7.0, about 7.2, about 7.5, about 8, about 8.5, or about 9.

In some embodiments, the methods of the present disclosure are carried out at a temperature of about 4 °C to about 37 °C. In some embodiments, the temperature is about 4 °C, about 5 °C, about 6 °C, about 8 °C, about 10 °C, about 12 °C, about 15 °C, about 18 °C, about 20 °C, about 22 °C, about 25 °C, about 28 °C, about 30 °C, about 32 °C, about 35 °C, or about 37 °C. In some embodiments, the temperature is at least about 4 °C, about 5 °C, about 6 °C, about 8 °C, about 10 °C, about 12 °C, about 15 °C, about 18 °C, about 20 °C, about 22 °C, about 25 °C, about 28 °C, or about 30 °C. In some embodiments, the temperature is at most about 37 °C, about 35 °C, about 32 °C, about 30 °C, about 28 °C, about 25 °C, about 22 °C, about 20 °C, about 18 °C, about 15 °C, about 12 °C, about 10 °C, about 8 °C, about 6 °C, about 5 °C, or about 4 °C. In some embodiments, the methods of the present disclosure are carried out at a temperature defined by any combination of the foregoing lower and upper endpoints, wherein the lower endpoint is less than the upper endpoint. Exemplary temperature ranges include about 4 °C to about 37 °C, about 4 °C to about 35 °C, about 4 °C to about 30 °C, about 4 °C to about 25 °C, about 5 °C to about 37 °C, about 10 °C to about 37 °C, about 10 °C to about 30 °C, about 15 °C to about 35 °C, about 15 °C to about 30 °C, about 20 °C to about 37 °C, about 20 °C to about 30 °C, about 22 °C to about 32 °C, about 25 °C to about 35 °C, about 25 °C to about 30 °C, and about 28 °C to about 32 °C. In some embodiments, the methods of the present disclosure are carried out at about 30 °C. In some embodiments, the methods of the present disclosure are carried out at about 37 °C.

In some embodiments, the One-Step DOS4 method provided herein is performed over a period of about 16 hours to about 48 hours. In some embodiments, the One-Step DOS4 method provided herein is performed for about 16 hours, about 18 hours, about 20 hours, about 24 hours, about 30 hours, about 36 hours, about 42 hours, or about 48 hours. In some embodiments, the duration is at least about 16 hours, about 18 hours, about 20 hours, about 24 hours, about 30 hours, or about 36 hours. In some embodiments, the duration is at most about 48 hours, about 42 hours, about 36 hours, about 30 hours, about 24 hours, about 20 hours, or about 18 hours. In some embodiments, the One-Step DOS4 method provided herein is performed for a duration defined by any combination of the foregoing lower and upper endpoints, wherein the lower endpoint is less than the upper endpoint. Exemplary durations include about 16 hours to about 48 hours, about 16 hours to about 42 hours, about 16 hours to about 36 hours, about 16 hours to about 30 hours, about 16 hours to about 24 hours, about 18 hours to about 48 hours, about 18 hours to about 36 hours, about 20 hours to about 48 hours, about 20 hours to about 30 hours, and about 24 hours to about 36 hours. In some embodiments, the One-Step DOS4 method provided herein is performed for about 16 hours.

In some embodiments, the One-Step DOS2 method provided herein is performed over a period of about 4 hours to about 48 hours. In some embodiments, the One-Step DOS2 method provided herein is performed for about 4 hours, about 5 hours, about 6 hours, about 8 hours, about 10 hours, about 12 hours, about 16 hours, about 24 hours, about 30 hours, about 36 hours, about 42 hours, or about 48 hours. In some embodiments, the duration is at least about 4 hours, about 5 hours, about 6 hours, about 8 hours, about 10 hours, about 12 hours, about 16 hours, or about 24 hours. In some embodiments, the duration is at most about 48 hours, about 42 hours, about 36 hours, about 30 hours, about 24 hours, about 16 hours, about 12 hours, about 10 hours, about 8 hours, about 6 hours, or about 5 hours. In some embodiments, the One-Step DOS2 method provided herein is performed for a duration defined by any combination of the foregoing lower and upper endpoints, wherein the lower endpoint is less than the upper endpoint. Exemplary durations include about 4 hours to about 48 hours, about 4 hours to about 36 hours, about 4 hours to about 24 hours, about 4 hours to about 12 hours, about 4 hours to about 6 hours, about 5 hours to about 48 hours, about 5 hours to about 24 hours, about 6 hours to about 48 hours, about 6 hours to about 24 hours, about 6 hours to about 12 hours, and about 8 hours to about 16 hours. In some embodiments, the One-Step DOS2 method provided herein is performed for about 6 hours. In some embodiments, the One-Step DOS2 method provided herein is performed for about 4 hours.

In certain embodiments, the antibody is a monoclonal antibody. In certain embodiments, the monoclonal antibody is a recombinant monoclonal antibody. In certain embodiments, the monoclonal antibody is derived from a hybridoma. In another embodiment, the antibody is a polyclonal antibody. In certain embodiments, the antibody is a therapeutic antibody. More particularly, the therapeutic antibody is selected from the group consisting of trastuzumab, cetuximab, bevacizumab, alemtuzumab, gemtuzumab, ibritumomab, rituximab, panitumumab, tremelimumab, nivolumimab, and pertuzumab.

The antibodies disclosed herein can be human, fully human, humanized, human engineered, non-human, and/or chimeric. The antibodies disclosed herein can be based on or derived from human, fully human, humanized, human engineered, non-human and/or chimeric antibodies. A non-human antibody can be humanized to reduce immunogenicity to humans, while retaining the specificity and affinity of the parental non-human antibody. Generally, a humanized antibody comprises one or more variable domains in which CDRs (or portions thereof) are derived from a non-human antibody, and FRs (or portions thereof) are derived from human antibody sequences. A humanized antibody optionally also comprises at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005); Klimka et al., Br. J. Cancer, 83:252-260 (2000) (describing the “guided selection” approach to FR shuffling); and Studnicka et al., U.S. Pat. No. 5,766,886.

Chimeric antibodies can refer to antibodies created through the joining of two or more antibody genes which originally encoded for separate antibodies. A chimeric antibody can comprise at least one amino acid from a first antibody and at least one amino acid from a second antibody, wherein the first and second antibodies are different. The antibodies disclosed herein can comprise antibody sequences from two or more different antibodies. In certain embodiments, the two or more different antibodies can be from the same species. For example, the species can be a bovine species, human species, or murine species. The two or more different antibodies can be from the same type of animal. For example, the two or more different antibodies can be from a cow. The two or more different antibodies can be from a human. Alternatively, the two or more different antibodies can be from different species. For example, the two or more different antibodies can be from a human species and bovine species. In another example, the two or more different antibodies can be from a bovine species and a non-bovine species. In another example, the two or more different antibodies can be from a human species and a non-human species.

In some aspects, the antibody or antibody conjugate can comprise an antibody or at least a portion of an antibody that is a human, fully human, humanized, human engineered, non-human, or chimeric antibody. In some aspects, the antibodies can comprise an antibody or at least a portion of an antibody that is a mammalian antibody. In some aspects, the antibodies can comprise an antibody or at least a portion of an antibody that is a non-mammalian antibody. In some aspects, the antibodies can comprise an antibody or at least a portion of an antibody that is cross-reactive.

Antibodies for use in the methods disclosed herein can be produced using any means known to those of ordinary skill in the art. General information regarding procedures for antibody production and labeling may be found, for example, in Antibodies: A Laboratory Manual, Cold Spring Harbor Laboratory, Chap. 14 (1988). Cell lines expressing antibodies can also be produced using any means known to those of ordinary skill in the art. For therapeutic purposes, chimeric, humanized, and completely human antibodies are useful for applications that include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application No. 184,187; Taniguchi, M., European Patent Application Publication No. 171,496; Morrison et al European Patent Application Publication No. 173,494; Neuberger et al PCT International Publication No. WO 86/01533; Cabilly et al U.S. Pat. No. 4,816,567; Cabilly et al European Patent Application Publication No. 125,023; Better et al., Science 240: 1041-1043 (1988); Liu et al., Proc. Natl. Acad. Sci. USA 84: 3439-3443 (1987); Liu et al., J. Immunol 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter, U.S. Pat. No. 5,225,539; Jones et al., Nature 321: 552-525 (1986); Verhoeyan et al., Science 239: 1534; and Beidler et al., J. Immunol. 141: 4053-4060 (1988).

Transgenic mice that are incapable of expressing endogenous immunoglobulin heavy and light chains genes, but that can express human heavy and light chain genes, may be used to produce human antibodies for use in the present teachings. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Thermo Fisher Scientific (Waltham, MA) can be engaged to provide human antibodies directed against a selected antigen using technology similar to that described above. Human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

The antibodies disclosed herein can be based on or derived from an antibody or antibody fragment from a mammal, bird, fish, amphibian or reptile. Mammals include, but are not limited to, carnivores, rodents, elephants, marsupials, rabbits, bats, primates, seals, anteaters, cetaceans, odd-toed ungulates and even-toed ungulates. The mammal can be a human, non-human primate, mouse, sheep, cat, dog, cow, horse, goat, or pig. Birds include, but are not limited to, albatrosses, hummingbirds, eagles, ostriches, cardinals, kiwis, and penguins. Fish can be cartilaginous fishes, ray-finned fishes, or lobe-fined fishes. Amphibians include, but are not limited to, newts, salamanders, frogs and toads. Examples of reptiles include, but are not limited to, turtles, squamates, crocodiles and tuataras. Squamates may include amphisbaenians, lizards and snakes.

In certain embodiments, the antibody conjugates described herein can be used to perform diagnostic imaging. The imaging technique can include whole body imaging for diagnostic purposes or local imaging at specific sites, such as but not limited to sites of tumor growth, in a quantitative manner to assess the progression of disease or host response to a treatment regimen. The imaging can be accomplished in vitro or in vivo by any suitable method known in the art. For example, and without wishing to be limiting, the diagnostic imaging technique can include immunohistochemistry, immunofluorescence staining, or a non-invasive (molecular) diagnostic imaging technology including, but not limited to: optical imaging; positron emission tomography (PET), wherein the detectable agent is an isotope such as ¹¹C, ¹³N, ¹⁵O, ¹⁸F, ⁶⁴Cu, ⁶²Cu, ¹²⁴I, ⁷⁶Br, ⁸²Rb and ⁶⁸Ga; or single photon emission computed tomography (SPECT), wherein the detectable agent is a radiotracer such as ^{99m}Tc, ¹¹¹In, ¹²³I, ²⁰¹Tl, ¹³³Xe, depending on the specific application.

In certain embodiments, the antibody conjugates are analyzed by gel electrophoresis. In certain embodiments, the antibody conjugates are analyzed by fluorescence microscopy. In certain embodiments, the antibody conjugates are analyzed by mass spectroscopy. In certain embodiments, the antibody conjugates are analyzed by flow cytometry. In certain embodiments, the antibody conjugates are analyzed by lateral flow assays. In certain embodiments, the antibody conjugates are analyzed by enzyme-linked immunosorbent assays (ELISA assays).

In certain embodiments, the antibody conjugates made by the methods disclosed herein can be used for treating a disease, disorder or condition in a subject in need thereof. In certain embodiments, the antibody conjugate is administered by parenteral administration comprising intravenous administration, subcutaneous administration, intraperitoneal administration, intramuscular administration, intravascular administration, intrathecal administration, intravitreal administration, or infusion. In certain embodiments, the antibody conjugate is administered by a microneedle device. In certain embodiments, antibody conjugate is administered by topical, oral or nasal administration. In certain embodiments, the disease is a cancer, a pathogenic infection, an inflammatory disease, an autoimmune disease or a metabolic disease. In certain embodiments, the subject is a mammal.

In certain embodiments, the antibody conjugates made by the methods disclosed herein can be used for diagnosing a disease, disorder or condition. The diseases can include, but are not limited to, cancer, including but not limited to breast cancer, prostate cancer, lung cancer, skin cancer, cancers of the reproductive tract, brain cancer, liver cancer, pancreatic cancer, stomach cancer, blood cancers (e.g., leukemia and lymphoma), sarcomas, melanomas, and the like, a pathogenic infection, an inflammatory disease, an autoimmune disease or a metabolic disease.

### Kits

Also provided herein are kits for carrying out the disclosed methods for one-step antibody activation. In certain embodiments, the kit includes a modified sugar substrate comprising a chemical handle, and an enzyme mixture comprising, consisting essentially of, or consists of a β-galactosidase and a mutant β-1,4-galactosyltransferase. In certain other embodiments, the kit includes a modified sugar substrate comprising a chemical handle, and an enzyme mixture comprising, consisting essentially of, or consists of an endoglycosidase and a mutant β-1,4-galactosyltransferase. In certain embodiments, the chemical handle comprises an azide group, an alkyne group, a cycloalkene group, a ketone group, an oxime group, a nitrile oxide group, or a vinyl group. In certain embodiments, the modified sugar substrate is chosen from UDP-GalNAz, UDP-GalKyne, UDP-Gal-Cyclopropene, UDP-GalKetone, UDP-Gal-Oxime, UDP-Gal-Nitrile oxide, UDP-GalVinyl, UDP-GalVinylSulfo, and UDP-GalVinylPhospho. In certain embodiments, the modified sugar substrate is chosen from:

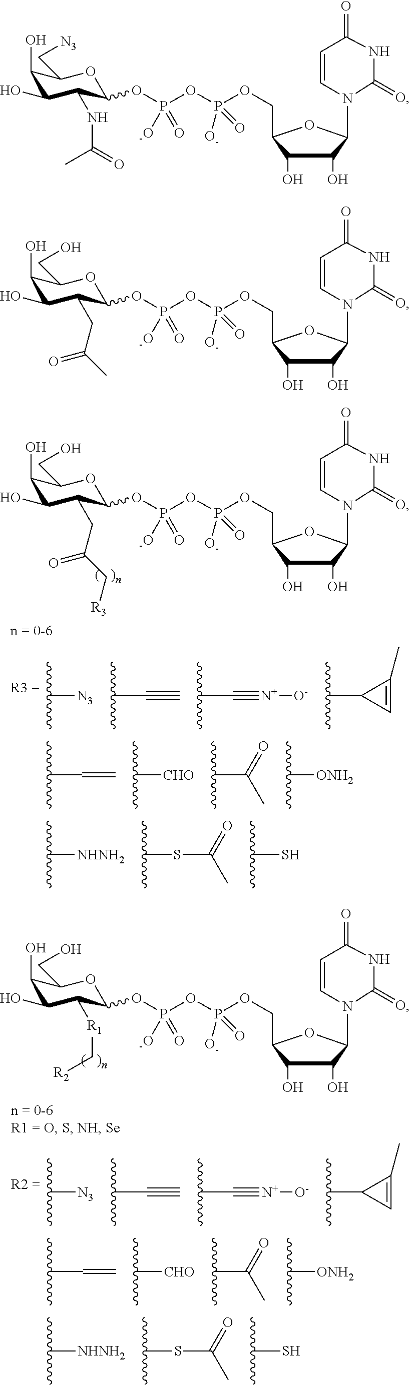

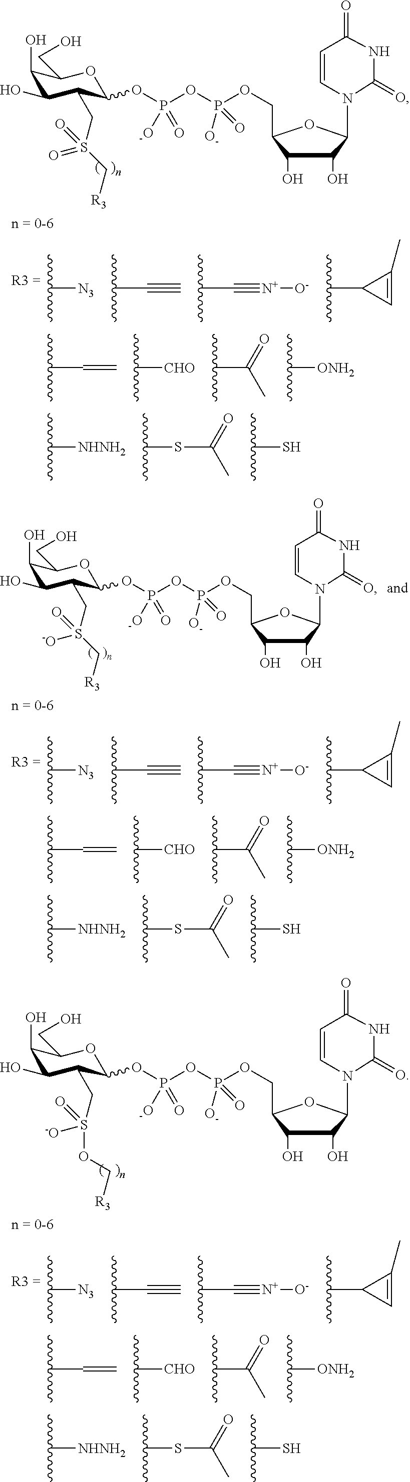

In certain preferred embodiments, the modified sugar substrate is UDP-GalNAz. In certain embodiments, the mutant β-1,4-galactosyltransferase is a Y289L, Y289N, Y289I, Y289F, Y289M, Y289V, Y289G, or Y289A mutant β-1,4-galactosyltransferase. In certain preferred embodiments, the mutant β-1,4-galactosyltransferase is a Y289L mutant β-1,4-galactosyltransferase. In certain embodiments, the endoglycosidase is chosen from Endo S, Endo A, Endo F, Endo M, Endo D, and Endo H or a combination thereof. In certain preferred embodiments, the endoglycosidase is Endo S or Endo S2.

The kit can include one or more additional items to carry out the claimed methods of antibody activation. In some examples, the kit further includes one or more buffers, salts, cofactors, reaction vessel(s), purification columns, collection tube(s), and/or instruction material. In certain embodiments, the cofactor is MnCl₂. In particular examples, the reaction vessel is a tube, microplate, or deep well plate.

### EXAMPLES

The following examples are provided to illustrate particular features of certain aspects of the disclosure, but the scope of the claims should not be limited to those features exemplified.

### Example 1: Protocols Used in the Characterization of the One-Step DOS4 and One-Step DOS2 Antibody Activation and Subsequent Antibody Conjugation Methods of the Present Disclosure

### Antibody Buffer Exchange

Antibodies were buffer exchanged into 50 mM Tris, pH 7.0 by performing 5 washes in a 50 kDa MWCO centrifugal filter. Centrifugation for each wash was carried out at 5,000 RCF until the retentate volume was <20% of the initial volume.

### Concentration Determination

Antibody concentrations were determined using a NanoDrop™ UV-Vis spectrophotometer. Absorbance at 280 nm was measured and concentration was calculated according to the Beer-Lambert Equation (E=210,000 M⁻¹cm⁻¹, 1=1 mm).

A280=ε×l×c

### Antibody Activation Workflow Modifications

Antibody glycans were activated with azide groups according to the procedures below.

Classic SiteClick™ Antibody Activation: 2 mg/mL buffer exchanged antibodies were mixed with 3.3 μg/mL β-Galactosidase (β-Gal) and incubated for 16 hrs at 37 °C. 0.14 mg/mL (Y289L)GalT, 10 mM MnCl₂, 2 mM UDP-GalNAz, and 50 mM Tris, pH 7.0 was then added and incubated overnight at 37 °C.

One-step DOS4 Antibody Activation: In the One-step DOS4 workflow of the present disclosure, 1 mg/mL buffer exchanged antibody, 0.015 mg/mL β-Galactosidase, 0.15 mg/mL (Y289L)GalT, 50 mM Tris, pH 7.0, 10 mM MnCl₂, 0.1 mM EDTA, 2 mM UDP-GalNAz were incubated at 37 °C for 16 hours.

“Classic” Two-step DOS2 Antibody Activation: 2 mg/mL buffer exchanged antibodies were mixed with 2 U/mL Endo S and incubated for 16 hrs at 30 °C. 0.14 mg/mL (Y289L)GalT, 10 mM MnCl₂, 2 mM UDP-GalNAz, and 50 mM Tris, pH 7.0 was then added and incubated overnight at 30 °C.

One-step DOS2 Antibody Activation: In the one step SiteClick DOS2 workflow of the present disclosure, 1 mg/mL buffer exchanged antibody, 2 U/μL Endo S, 0.15 mg/mL (Y289L)GalT, 50 mM Tris, pH 7.0, 10 mM MnCl₂, 0.1 mM EDTA, 2 mM UDP-GalNAz were incubated at 30 °C for 6 hours.

### Removal of Free UDP-GalNAz

Free UDP-GalNAz was removed from the antibody activation reactions by buffer exchange as described above.

sDIBO-AF488 Labeling and Quantification of Degree of Substitution (DOS)

To measure the degree of substitution of the azide activated antibodies, Alexa Fluor™ 488 dye was conjugated with an sDIBO reactive group (sDIBO-AF488) and the antibody and dye concentrations were measured using a NanoDrop™ UV-Vis spectrophotometer. 1 mg/mL azide activated antibodies were mixed with 0.2 mM sDIBO-AF488 and incubated overnight at room temperature. Free dye was removed by buffer exchange as described above. Antibody concentration and degree of labeling was quantified using the Beer-Lambert Equation using a correction factor for sDIBO-AF488.

$$Protein concentration ( M ) = \frac{[ A 280 - ( A 495 \times 0.134 ) ]}{210000}$$

$$DOL = \frac{A 495}{710 \; 00 \times Protein concentration ( M )}$$

### SDS-PAGE Analysis

Samples were prepared for SDS-PAGE analysis by mixing at the following final concentrations; 0.1 mg/mL sDIBO-AF488 antibody conjugate, 50 mM DTT, 1× Sample Loading Buffer. Samples were heated at 70 °C for 10 min then cooled on ice for 5 min. 1 μg of sDIBO-AF488 antibody conjugate per lane was loaded onto NuPAGE 4-12% Bis-Tris gels and run at 200 V for 50 min in 1×MOPS SDS running buffer. The gel was then imaged for AF488 fluorescence using an iBright™ imager (Thermo Fisher Scientific). Total protein was measured by staining with SimplyBlue™ SafeStain (Thermo Fisher Scientific) and iBright imaging.

### Flow Cytometry

Frozen peripheral blood mononuclear cells (PBMCs) purchased from Discovery Life Sciences were thawed at 37 °C and resuspended in 10 mL RPMI (Thermo Fisher Scientific 22400-071). Wash steps were carried out by resuspending cells at the indicated concentration, centrifuging 500 RCF for 5 min and discarding the supernatant. PBMCs were then washed with PBS (Thermo Fisher Scientific 70011-044) and resuspended at 1 million cells/mL. Cells were stained with 1 μL of Live/Dead Fixable Green (ThermoFisher L23101) per 1 mL for 20 min on ice. Cells were then washed with 10 mL Flow Cytometry Stain Buffer (FSB) (ThermoFisher 00-4222-26) and resuspended at 10 million cells/mL. 0.1 mL of cells were removed for each sample and the indicated amount was conjugated using Classic SiteClick, One-Step DOS4, or One-Step DOS2 activated antibodies or co-stain antibodies were added for surface staining. Surface staining was carried out for 30 min on ice. Cells were washed with FBS then PBS, resuspended in 0.5 mL PBS, and analyzed on an Attune™ NxT flow cytometer. Data was analyzed using FlowJo™ v 10.10 software.

TABLE 1
Co-stain antibodies

| | | | Stain amount |
|---|---|---|---|
| Antibody conjugate | Supplier | Catalog # | per test |
| CD3 eF450 | Thermo Fisher | 48-0037-42 | 5 μL (1 μg) |
| | Scientific | | |
| CD19 eF450 | Thermo Fisher | 48-0198-42 | 5 μL (0.125 μg) |
| | Scientific | | |

### Mass Spectrometry

Intact protein LC-MS of all samples were performed on a Vanquish™ Horizon UHPLC system (Thermo Fisher Scientific) coupled to an Orbitrap Exploris™ 240 mass spectrometer (Thermo Fisher Scientific). About 1.0-1.5 μg of sample was injected from concentrations between 0.30-1.0 mg/mL into a MAbPac SEC-1 column (300 mm×7.8 mm) in PBS (Thermo Scientific). Antibodies were eluted using an isocratic gradient of 100% 50 mM NH₄OAc over 10 minutes at 200 μL/min after a pre-injection equilibration of 5 minutes. For all samples, the Orbitrap Exploris™ 240 was operated in intact protein application mode for single MS acquisition through the Xcalibur™ software (Thermo Fisher Scientific). The spray voltage was set to 3,800 V, the ion transfer tube temperature to 275 °C, the vaporizer temperature to 250 °C, the RF lens to 200%, the in-source energy at 120 V, with a normalized AGC target of 300% and a maximum injection time of 200 ms. The MS scans were averaged every 5 μscans, acquired over the range of 2,500-8,000 m/z at a resolution set to 30,000. The raw MS data of the intact protein spectra were deconvoluted with the Sliding Windows and ReSpect™ algorithms through the software BioPharma Finder™ 5.2 (Thermo Fisher Scientific). The parameters for component detection included: restricted time set to the time where the intact protein peak eluted from the column; m/z range of 2,500-8,000; a target average spectrum offset of 1; Sliding Windows merge tolerance of 30.0 ppm and a max RT gap of 1.00 minute; deconvolution output mass range of 130,000-180,000 Da that encompasses the desired protein mass and mass tolerance of 20.0 ppm Example 2: One-step DOS4 workflow results in equivalent labeling as the Classic workflow

In traditional site-specific enzymatic antibody activation methods, β-galactosidase (β-Gal) performs the first enzymatic transformation of the antibody glycan to be modified, cleaving terminal galactose residues. The activity of β-Gal is reported to be optimal at pH 6.0 and reduced at pH 7.0. In order to achieve the One-Step DOS4 antibody activation method, we assessed the ability of β-Gal to perform the galactose cleavage reaction at pH 7.0, to allow the simultaneous use of β-Gal and the mutant β-1,4-galactosyltransferase (GalT). We confirmed previous observations that β-Gal shows reduced activity at pH 7.0 relative to pH 6.0 when cleaving a small molecule substrate, DDAO-galactoside (FIG. 4A). β-Gal cleavage of DDAO-galactoside was measured by an increase in fluorescence (ex: 640/10, em: 665/10) and compared to a standard curve of DDAO at the indicated pH to quantify the amount of DDAO cleaved. The rate of DDAO-galactoside cleavage was approximately four-fold slower at pH 7.0 than at pH 6.0 (FIG. 4A). Given that the rate of β-Gal cleavage was slower at pH 7.0 than at pH 6.0, we reasoned that a longer incubation time may still allow 1-Gal to perform the necessary cleavage reaction on the antibody under conditions compatible with the mutant (Y289L)GalT. After 16 hr incubation at pH 7.0, (3-Gal showed the same activity in the DDAO-galactoside cleavage assay, indicating that the enzyme remained active throughout an overnight reaction incubation (FIG. 4B). We then tested the pH dependence and verified activity on antibody glycan galactose cleavage by measuring the formation of antibody GOF/GOF glycan species on a NIST mAb, described in Agnew et al, Anal. Bioanal. Chem. 413:4989 (2021) (FIG. 4C). Surprisingly, the cleavage of antibody glycan terminal galactose groups occurred at approximately the same rate at pH 6.0 and pH 7.0. Given the surprisingly similar activity of 3-Gal on antibody glycan galactose cleavage, we determined that the β-Gal and mutant (Y289L)GalT reactions can advantageously be carried out in a single step, resulting in the One-Step DOS4 antibody activation method of the present disclosure.

Previous one-step labeling methods have been shown to be sensitive to antibody glycan fucosylation state and highly fucosylated antibodies must first have the fucosyl groups cleaved before the labeling reaction can be performed (Yang et al, bioRxiv 2022.09.04.506510; doi: https://doi.org/10.1101/2022.09.04.506510). To verify that the One-Step DOS4 antibody activation workflow of the present disclosure is not sensitive to antibody fucosylation state, we characterized the antibody glycan composition in β-Gal cleavage and GalNAz modification (e.g., antibody activation). The NIST mAb shows a highly fucosylated glycan, with a large majority of glycans containing a fucose. β-Gal cleavage of these glycans was measured and quantified by mass spectrometry (FIGS. 5A, 5B). Glycan distribution before and after β-Gal digestion showed that the fucosylated glycans can be cleaved by β-Gal under the conditions of the One-Step DOS4 antibody activation method. In addition, the complete transformation of the NIST mAb with GalNAz activation in the One-Step DOS4 workflow was measured by mass spectrometry (FIGS. 5C, 5D). Surprisingly, the NIST-mAb was nearly completely modified, e.g., activated, to a GalNAz degree of substitution of 4 despite the highly fucosylated state, contrary to prior one-step glycosylation methods.

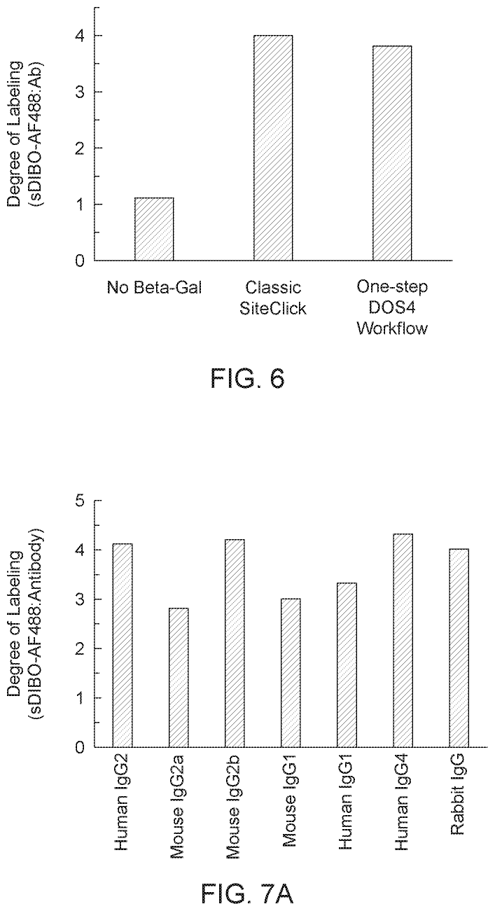

The above data indicates that the One-Step DOS4 antibody activation method of the present disclosure can modify antibodies similarly to the classic two-step SiteClick™ antibody activation method. The classic two-step SiteClick™ method results in addition of up to 4 labels per antibody, corresponding to the four termini of a typical IgG Fc glycan. To confirm equivalent functionality of the One-Step antibody activation method in antibody conjugation with a detectable label, the attachment of an Alexa Fluor™ 488 fluorophore comprising a strained-ring alkyne functional group, sDIBO, (sDIBO-AF488) was compared using antibodies prepared using the classic two-step method or the One-Step DOS4 method of the present disclosure (see FIG. 2). A similar degree of labeling of just under 4 was observed using either method as measured by UV-Vis spectroscopy (FIG. 6). Mass spectrometry analysis of the labeled antibody using the One-Step DOS4 method showed that most antibodies in the sample had a GalNAz degree of substitution (DOS) of 4, while a minority had only 2 or 3 GalNAz glycans attached (FIGS. 4C, 4D).

### Example 3: One-Step DOS4 Workflow Allows Labeling Across a Variety of Antibody Isotypes

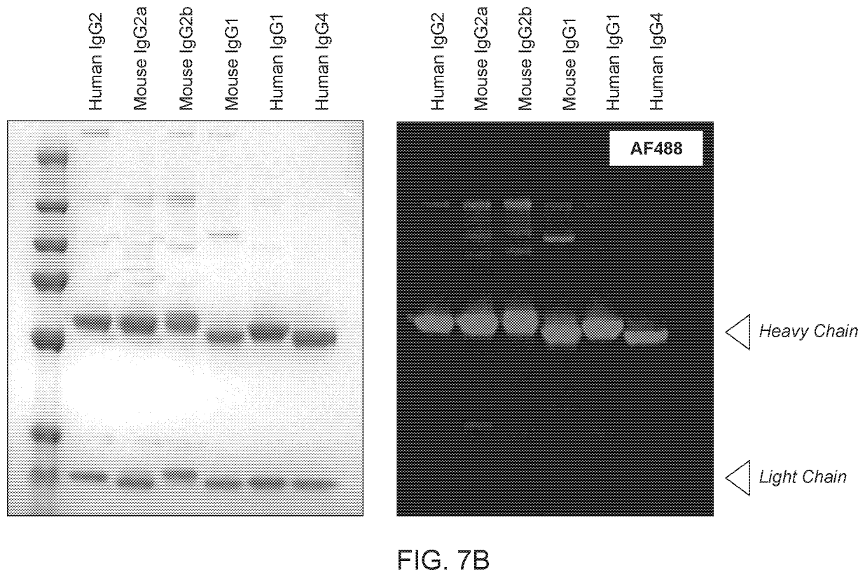

A major benefit of traditional SiteClick™ site-specific conjugation is that antibodies can be reproducibly modified regardless of their isotype. To demonstrate the universality of the new One-Step DOS4 method, the one-step antibody activation method was performed on a variety of antibody isotypes, shown in Table 2, and the degree of labeling by reaction with sDIBO-AF488 was quantified by UV-Vis spectroscopy. The average DOL across all isotypes using the one-step DOS4 method was ~3.7, confirming that the One-Step DOS4 method can reproducibly activate and label antibodies regardless of isotype (FIG. 7A). We confirmed that the labeling was specific to the antibody heavy chain, where the glycan is attached, by SDS-PAGE and Coomassie staining (total protein) or AF488 fluorescence (FIG. 7B).

TABLE 2
Antibodies used in isotype testing

| Isotype | Antibody |
|---|---|
| Human IgG1 | Rituximab |
| Human IgG2 | Tremelimumab |
| Human IgG4 | Nivolumab |
| Mouse IgG1 | CD28 clone 28.2 |
| Mouse IgG2a | CD3 OKT3 |
| Mouse IgG2b | CD4 OKT4 |
| Rabbit IgG | Alexa Fluor ™ 488 (AF488) Polyclonal |

### Example 4: One-Step DOS4 Modified Antibodies are Functional in Flow Cytometry

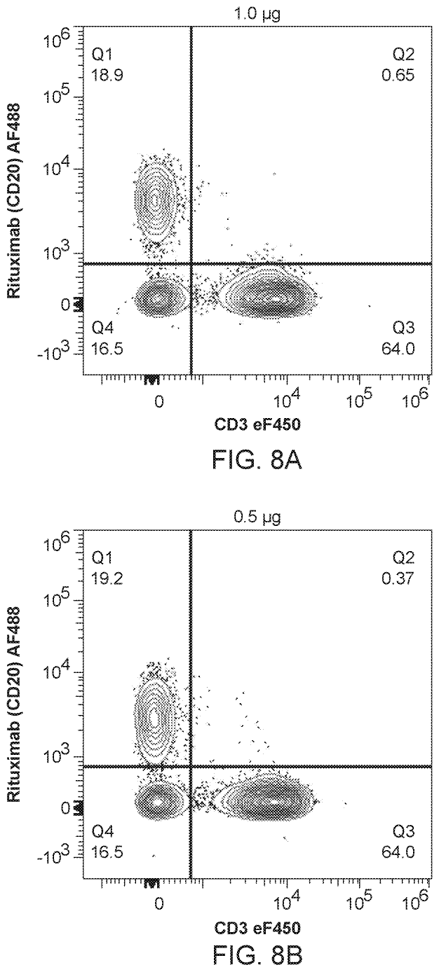

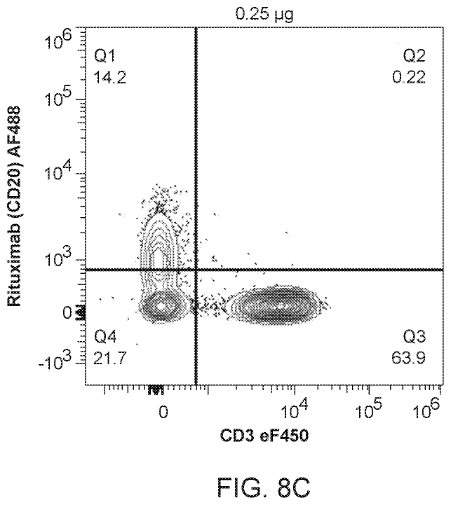

Antibodies activated and modified by the One-Step DOS4 method described herein can be used in a variety of applications. In order to demonstrate functionality of the modified antibodies, we labeled Rituximab (anti-CD20) with sDIBO-AF488 using the One-Step DOS4 workflow and analyzed human peripheral blood mononuclear cells (PBMCs) by flow cytometry (FIGS. 8A-8C). Rituximab stained cells were well separated from the negative population and were titratable. The labeled antibody stained B-cells specifically based on the mutually exclusive staining of CD3 eF450.

### Example 5: One-Step DOS2 Workflow Results in Equivalent Labeling as the Existing Two-Step Method

When adapting the DOS2 workflow to a single step, it was unclear whether or not Endo S could cleave a GalNAz modified antibody. If Endo S is able to cleave the GalNAz activated antibody then it would be unlikely that a single-step workflow would be successful as the Endo S would need to be purified away prior to the mutant GalT-mediated modification of the antibody. In order to test whether Endo S can cleave the GalNAz activated antibody, we modified a CD28 clone 28.2 mAb using the One-Step workflow as described in FIG. 3 (Step 310) and then re-treated the modified antibody with Endo S and incubated overnight. Surprisingly, we measured by mass spectrometry identical spectra with or without the Endo S re-treatment indicating that Endo S was unable to cleave GalNAz from the activated antibody (FIG. 9A). Additionally, we re-treated an antibody with Endo S after complete labeling with sDIBO-AF488 and demonstrated retention of the fluorescent label by UV-Vis spectroscopy (FIG. 9B).

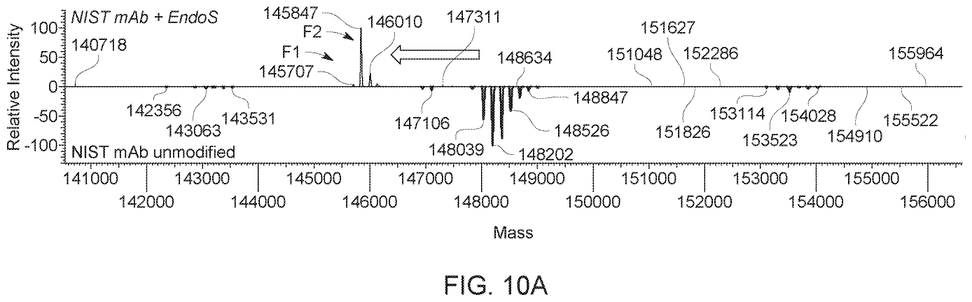

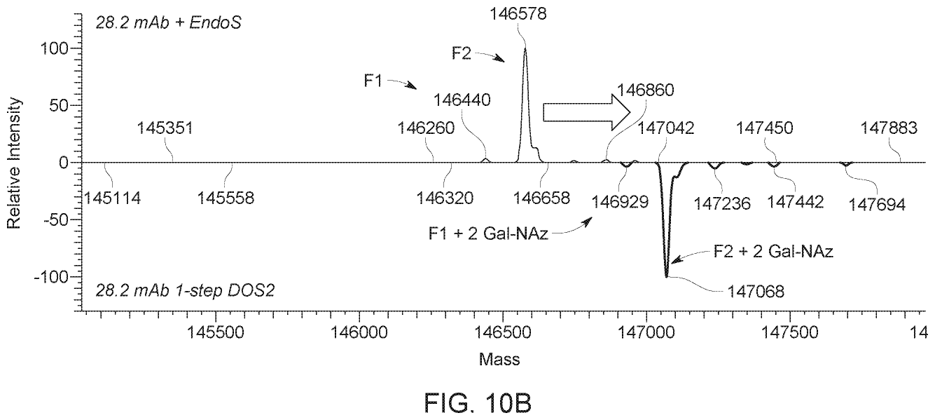

We also assessed whether the fucosylation state of the antibody affects its activity as a target of the one-step DOS2 workflow of the present disclosure. We digested the NIST-mAb, a highly fucosylated antibody, with Endo S similarly to the One-Step DOS2 workflow and measured mass distribution by mass spectrometry (FIG. 10A). All fucosylated species in the unmodified antibody were cleaved by Endo S to produce lower mass peaks indicating that Endo S was able to cleave antibody glycans regardless of their fucosylation state. Additionally, we measured the differences in CD28 clone 28.2 mAb in a One-Step DOS2 workflow with and without (Y289L)GalT (FIG. 10B). The mass difference of the modified antibody in these reactions indicated that two GalNAz glycans were added to each antibody regardless of the fucosylation state.

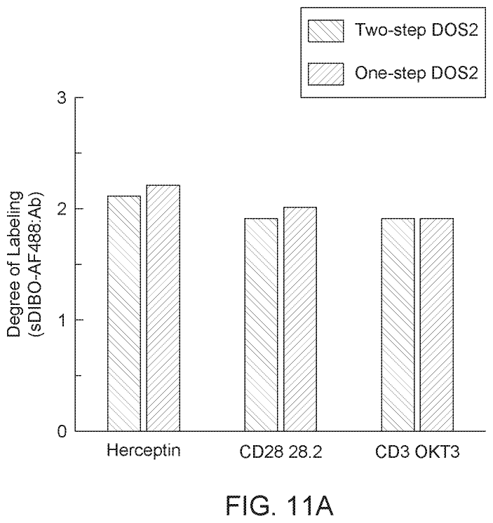

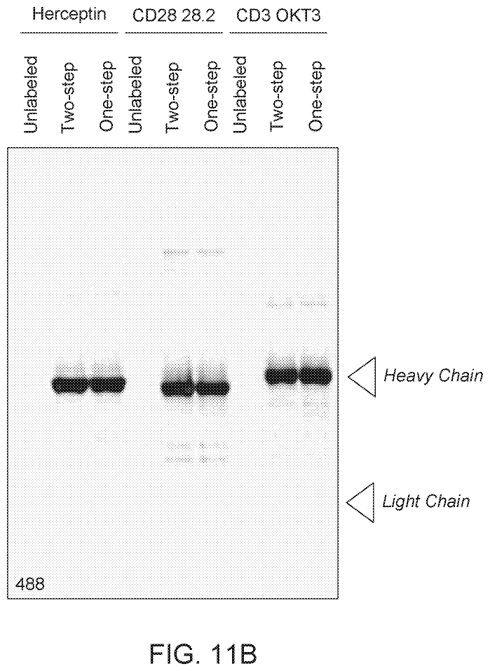

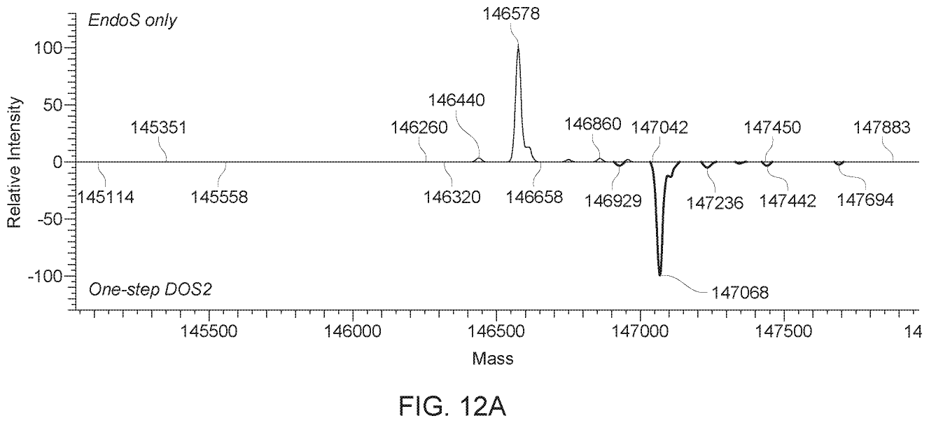

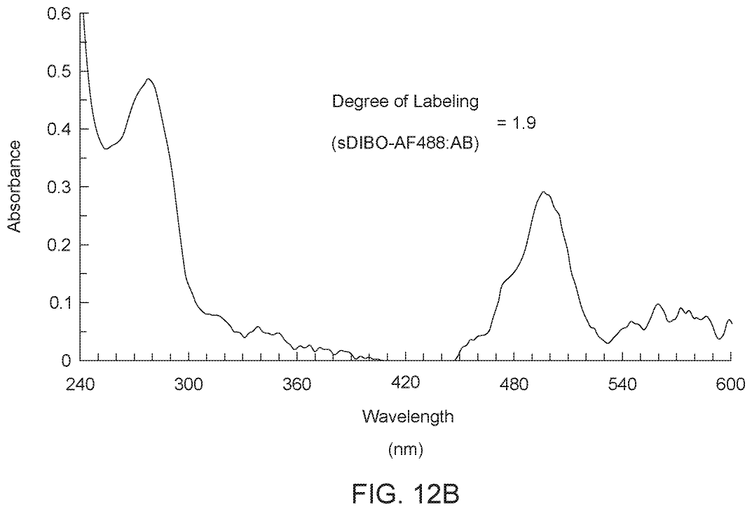

The DOS2 antibody modification workflow has previously been carried out as a two-step reaction. In order to assess if the One-Step DOS2 workflow of the present disclosure performs equivalently, we modified three different antibodies using a one-step or two-step DOS2 workflow and measured the degree of labeling (DOL) by attachment of sDIBO-AF488 and UV-Vis spectroscopy (FIG. 11A). We observed nearly identical AF488 DOL in antibodies prepared in the one-step or two-step workflows. These antibodies were specifically labeled in the heavy chain, which carries the amino acid the antibody glycan is attached to (FIG. 11B). To verify a DOS2 product was formed by the one-step activation reaction of the present disclosure, we measured the mass difference in an Endo S cleaved antibody CD28 clone 28.2 and a One-Step DOS2 prepared antibody. The difference in mass peaks corresponds to the addition of exactly two GalNAz glycans (FIG. 12A). This DOL was verified by attachment of sDIBO-AF488 and measurement by UV-Vis spectrometry (FIG. 12B). This detailed comparison of mass spectrometry and UV-Vis measurements additionally validated the use of sDIBO-AF488 labeling and spectrophotometry as a method to quantify DOL.

### Example 6: One-Step DOS2 Workflow Allows Labeling Across a Variety of Antibody Isotypes

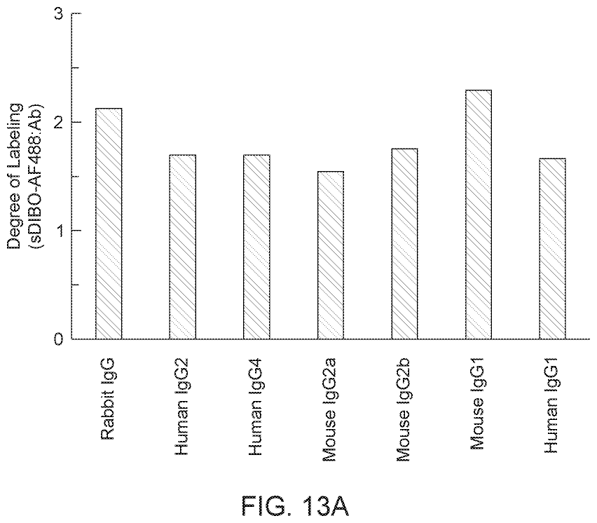

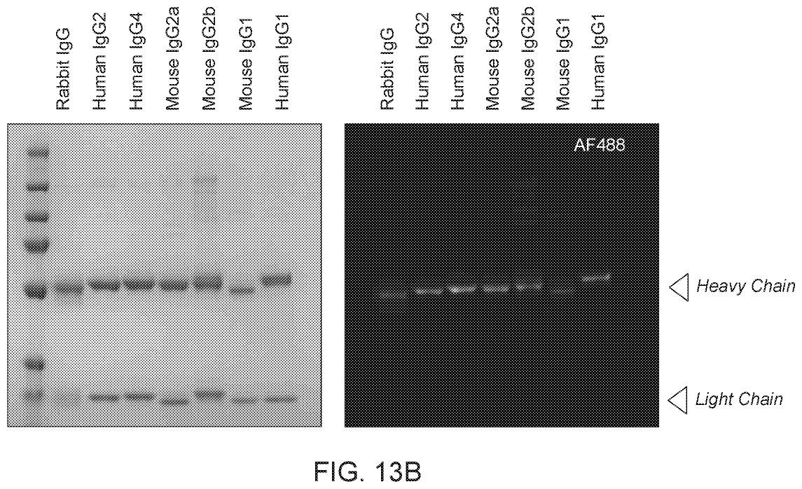

A major advantage of the SiteClick™ site-specific antibody modification is the reproducibility of the method across antibody isotypes. We demonstrated herein the reproducibility of the One-Step DOS2 workflow of the present disclosure by attaching sDIBO-AF488 to a variety of antibody isotypes (FIG. 13A and Table 2). This One-Step DOS2 workflow reproducibly yielded a DOL averaging ~1.8. Labeling was specific for the antibody heavy chain across all isotypes tested (FIG. 13B).

### Example 7: One-Step DOS2 Modified Antibodies are Functional in Flow Cytometry

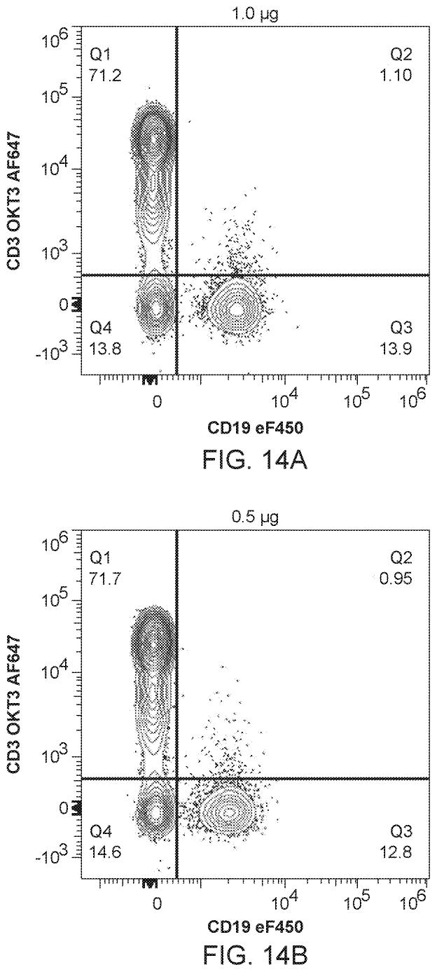

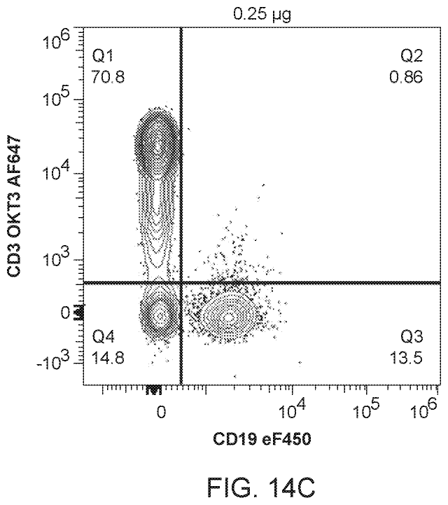

The functionality of the antibody conjugates produced from the One-Step DOS2 workflow of the present disclosure was tested in flow cytometry (FIGS. 14A-14C). AF488 labeled OKT3 cells labeled using the One-Step DOS2 workflow, identified T-cells in human PBMCs by mutually exclusive staining with CD19 eF450. Positive signal from OKT3-AF488 was well separated from negative events and titratable.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1. A method comprising:
contacting an antibody with a modified sugar substrate comprising a first chemical handle, a mutant β-1,4-galactosyltransferase, and a β-galactosidase in a single step to form an activated antibody.

2. A method comprising:
contacting an antibody with a modified sugar substrate comprising a first chemical handle, a mutant β-1,4-galactosyltransferase, and an endoglycosidase in a single step to form an activated antibody.

3. The method of claim 1, further comprising:
contacting the activated antibody with a biologically or pharmaceutically active molecule that comprises a second chemical handle that reacts with the first chemical handle to form an antibody conjugate.

4. The method of claim 1, wherein the first chemical handle comprises an azide group, an alkyne group, a cycloalkene group, a ketone group, an oxime group, a nitrile oxide group, or a vinyl group.

5. The method of claim 1, wherein the modified sugar substrate is chosen from UDP-GalNAz, UDP-GalKyne, UDP-Gal-Cyclopropene, UDP-GalKetone, UDP-Gal-Oxime, UDP-Gal-Nitrile oxide, UDP-GalVinyl, UDP-GalVinylSulfo, and UDP-GalVinylPhospho.

6-7. (canceled)

8. The method of claim 3, wherein the second chemical handle comprises an alkyne-reactive group, an azide-reactive group, a Diels-Alder diene, a Diels-Alder dienophile, a thiol, a straight chain or branched C₁-C₁₂ carbon chain bearing a carbonyl group, a —NR¹NH₂ (hydrazide), —NR¹(C═O)NR²NH₂ (semicarbazide), —NR¹(C═S)NR²NH₂ (thiosemicarbazide), —(C═O)NR¹NH₂ (carbonylhydrazide), —(C═S)NR¹NH₂ (thiocarbonylhydrazide), —(SO₂)NR¹NH₂ (sulfonylhydrazide), —NR¹NR²(C═O)NR³NH₂ (carbazide), —NR¹NR²(C═S)NR³NH₂ (thiocarbazide), or —ONH₂ (aminooxy), wherein each R¹, R², and R³ is independently H or alkyl having 1-6 carbons.

9. The method of claim 3, wherein the second chemical handle comprises a terminal alkyne or a strained ring alkyne.

10. (canceled)

11. The method of claim 1, wherein the mutant β-1,4-galactosyltransferase is a Y289L, Y289N, Y289I, Y289F, Y289M, Y289V, Y289G, or Y289A mutant β-1,4-galactosyltransferase.

12. (canceled)

13. The method of claim 2, wherein the endoglycosidase is chosen from Endo S, Endo A, Endo F, Endo M, Endo D, Endo H, or a combination thereof.

14. (canceled)

15. The method of claim 3, wherein the biologically or pharmaceutically active molecule is chosen from a fluorescent dye, an enzyme, a radiolabel, a metal chelator, and a detectable substrate.

16. The method of claim 3, wherein the biologically or pharmaceutically active molecule is chosen from a therapeutic agent, a nucleic acid, a polynucleotide, an oligonucleotide, a protein, a peptide, a sugar, an oligosaccharide, a bead, and a lipid nanoparticle.

17. The method of claim 1, wherein the degree of labeling is about 4.

18. The method of claim 2, wherein the degree of labeling is about 2.

19. The method of claim 1, wherein the antibody is fucosylated before being contacted with the modified sugar substrate, the mutant β-1,4-galactosyltransferase, and the β-galactosidase or the endoglycosidase.

20. The method of claim 1, wherein the antibody is defucosylated before being contacted with the modified sugar substrate, the mutant β-1,4-galactosyltransferase, and the β-galactosidase or the endoglycosidase.

21-30. (canceled)

31. An antibody conjugate made by the method of claim 3.

32. A kit comprising:
an enzyme mixture comprising a β-galactosidase and a mutant β-1,4-galactosyltransferase; and
a modified sugar substrate comprising a chemical handle; or
an enzyme mixture comprising an endoglycosidase and a mutant β-1,4-galactosyltransferase; and
a modified sugar substrate comprising a chemical handle.

33. (canceled)

34. The kit of claim 32, wherein the chemical handle comprises an azide group, an alkyne group, a cycloalkene group, a ketone group, an oxime group, a nitrile oxide group, or a vinyl group.

35. The kit of claim 32, wherein the modified sugar substrate is chosen from UDP-GalNAz, UDP-GalKyne, UDP-Gal-Cyclopropene, UDP-GalKetone, UDP-Gal-Oxime, UDP-Gal-Nitrile oxide, UDP-GalVinyl, UDP-GalVinylSulfo, and UDP-GalVinylPhospho.

36-37. (canceled)

38. The kit of claim 32, wherein the mutant β-1,4-galactosyltransferase is a Y289L, Y289N, Y289I, Y289F, Y289M, Y289V, Y289G, or Y289A mutant β-1,4-galactosyltransferase.

39-45. (canceled)
